SEQUENCE LISTING (8 sequences)

SEQ ID NO: 1 - protein, 540 aa, Streptococcus pyogenes
ESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 2 - protein, 540 aa, Streptococcus pyogenes
ESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNLAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAFEWWRKVIDERDVKLSKEINVNISGSTLSPYGSITYK

SEQ ID NO: 3 - DNA, 35 bp, Artificial Sequence
  Synthetic - oligonucleotide synthesized in laboratory
caccatgcgacaaacacaggtctcaaatgatgttg

SEQ ID NO: 4 - DNA, 35 bp, Artificial Sequence
  Synthetic - oligonucleotide synthesized in laboratory
tttctgagtaggtgtaccgttatggtagttaatgg

SEQ ID NO: 5 - protein, 229 aa, Streptococcus pyogenes
RQTQVSNDVVLNDGASKYLNEALAWTFNDSPNYYKTLGTSQITPALFPKAGDILYSKLDELGRTRTARGTLTYANVEGSYGVRQSFGKNQNPAGWTGNPNHVKYKIEWLNGLSYVGDFWNRSHLIADSLGGDALRVNAVTGTRTQNVGGRDQKGGMRYTEQRAQEWLEANRDGYLYYEAAPIYNADELIPRAVVVSMQSSDNTINEKVLVYNTANGYTINYHNGTPTQK

SEQ ID NO: 6 - DNA, 32 bp, Artificial Sequence
  Synthetic - oligonucleotide synthesized in laboratory
aaaggcgcccgccaaaatttgacttatgccaa

SEQ ID NO: 7 - DNA, 38 bp, Artificial Sequence
  Synthetic - oligonucleotide synthesized in laboratory
aaactcgagctatttggaaaatgataattgaagtaaca

SEQ ID NO: 8 - protein, 827 aa, Streptococcus pyogenes
RQNLTYANEIVTQRPKRESVISDKSNFPVISPYLASVDFGERKTPLPTPDKGVKVTTEQSIAQVRKGPEERPYTVTGKITSVINGWGGYGFYIQDSEGIGLYVYPQKDLGYSKGDIVQLTGTLTRFKGDLQLQQVTAHKKLELSFPTSVKEAVISELETTTPSTLVKLSHVTVGELSTDQYNNTSFLVRDDSGKSIVVHIDHRTGVKGADVVTKISQGDLINLTAILSIVDGQLQLRPFSLEQLEVVKKVTSSNSDASSRNIVKIGEIQGASHTSPLLKKAVTVEQVVVTYLDDSTHFYVQDLNGDGDLATSDGIRVFAKNAKVQVGDVLTISGEVEEFFGRGYEERKQTDLTITQIVAKAVTKTGTAQVPSPLVLGKDRIAPANIIDNDGLRVFDPEEDAIDYWESMEGMLVAVDDAKILGPMKNKEIYVLPGSSTRPLNNSGGVLLPANSYNTDVIPVLFKKGKQIIKAGDSYKGRLAGPVSYSYGNYKVFVDDSKNMPSLMDGHLKPEKTNLQKDLSKLSIASYNIENFSANPSSTKDEKVKRIAESFIHDLNAPDIIGLIEVQDNNGPTDDGTTDATQSAQRLIDAIKKLGGPTYRYVDIAPENNVDGGQPGGNIRTGFLYQPERVSLSDKPKGGARDALTWVNGELNLSVGRIDPTNAAWKDVRKSLAAEFIFQGRKVVVVANHLNSKRGDNALYGCVQPVTFKSEQRRHVLANMLAQFAKEGAKHQANIVMLGDFNDFEFTKTIQLIEEGDMVNLVSRHDISDRYSYFHQGNNQTLDNILVSRHLLDHYEFDMVHVNSPFMEAHGRASDHDPLLLQLSFSK

(19) United States
(12) Patent Application Publication
(10) Pub. No.: US 2020/0181207 A1
(43) Pub. Date: Jun. 11, 2020

(54) ANALYTICAL AND THERAPEUTIC METHODS AND COMPOSITIONS, AND USES THEREOF

(71) Applicant: AUCKLAND UNISERVICES LIMITED, Auckland (NZ)

(72) Inventors: Nicole J MORELAND, Auckland (NZ); Paul Gary YOUNG, Auckland (NZ); Thomas PROFT, Auckland (NZ)

(73) Assignee: AUCKLAND UNISERVICES LIMITED, Auckland (NZ)

(21) Appl. No.: 16/608,651

(22) PCT Filed: Apr. 26, 2018
(86) PCT No.: PCT/NZ2018/050057

§ 371 Date: Oct. 25, 2019

(30) Foreign Application Priority Data
Apr. 26, 2017 (NZ) 731324
Oct. 16, 2017 (NZ) 736448

(51) Int. Cl. C07K 14/315; G01N 33/569

(52) U.S. Cl. CPC C07K 14/315; G01N 2800/347; G01N 2333/315; G01N 33/56944

(57) ABSTRACT

The invention generally relates to compositions and methods for treating, detecting and assisting in the diagnosis of Streptococcus pyogenes infection, of rheumatic fever or of poststreptococcal glomerulonephritis (PSGN), and compositions and methods for assessing the propensity for developing rheumatic fever or PSGN in subjects in need thereof. Recombinant polypeptides, including recombinant Streptococcus pyogenes SpnA polypeptides, and compositions comprising such polypeptides, for use in such methods are also provided.

### TECHNICAL FIELD

The invention generally relates to compositions and methods for treating, detecting and assisting in the diagnosis of Streptococcus pyogenes infection, rheumatic fever or poststreptococcal glomerulonephritis (PSGN), and compositions and methods for assessing the propensity for developing rheumatic fever or PSGN.

### BACKGROUND ART

Group A Streptococcus (GAS, Streptococcus pyogenes) cause or are associated with a number of diseases of varying severity—from mild skin infections and pharyngitis to severe invasive diseases and post-infection immune sequelae such as rheumatic fever or poststreptococcal glomerulonephritis (PSGN). Acute rheumatic fever and associated rheumatic heart disease is the major cause of acquired heart disease in the developing world.

Streptococcal serology is crucial for diagnosis of post-infection immune sequelae as these sequelae occur several weeks after GAS infection at a time when diagnostic culture of the causative bacteria is usually no longer possible. Generally, current clinical practice involves the measurement of antibody titres to two antigens, streptolysin-O (SLO) and deoxyribonuclease-B (DNaseB). The serological tests are referred to as anti-streptolysin-O (ASO) and anti-deoxyribonuclease-B (ADB), respectively. ASO titres are commonly measured using nephelometric or turbidimetric assays, and values are normally reported as international units per millilitre (IU/mL). ADB tests are less standardised, as in contrast to ASO, no reference sera are available for DNaseB. ADB titres are usually measured using an enzyme inhibition assay, where the inhibition of DNaseB activity by sera is detected using a coloured dye.

Confoundingly, marked variability in the immunokinetics of ASO and ADB antibody response has been reported, with a majority of children suffering GAS pharyngitis exhibiting elevated ASO and ADB titres (compared to pre-infection levels) for more than one year following infection [1]. Hence, a substantial risk of false positive diagnoses exists.

A need remains for more effective methods of identifying subjects who have or have recently had GAS infection, and for identifying subjects having a propensity for developing rheumatic fever or PSGN, and for more effective detection and diagnosis of rheumatic fever or PSGN.

It is an object of the invention to address the foregoing problems, or to provide methods and compositions for detecting and assisting in the diagnosis of rheumatic fever or poststreptococcal glomerulonephritis, or for assessing propensity for developing rheumatic fever or poststreptococcal glomerulonephritis, and/or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

### SUMMARY OF THE INVENTION

These and other objects are achieved in accordance with one or more aspects of the present invention.

Accordingly, in one aspect, the invention relates to a method for detecting recent exposure to Streptococcus pyogenes in a subject, the method comprising:
providing a biological sample from the subject that is capable of or suspected of containing antibodies specific for one or more Streptococcus pyogenes antigens;
contacting the biological sample with two or more populations of Streptococcus pyogenes antigen, wherein each of the two or more populations of Streptococcus pyogenes antigen is capable of binding antigen-specific antibodies present in the biological sample to form two or more populations of antigen:antigen-specific antibody complexes if the antigen-specific antibodies are present in the biological sample; and
detecting the complexes, wherein an increase in detection of one or more complexes above a threshold value is indicative of a recent exposure to Streptococcus pyogenes in the subject.

In another aspect, the present invention relates to a method for detecting or diagnosing rheumatic fever or poststreptococcal glomerulonephritis (PSGN) including acute poststreptococcal glomerulonephritis (APSGN) in a subject, or an increased likelihood of developing rheumatic fever or PSGN in a subject, the method comprising:
i) providing a biological sample from the subject that is capable of or suspected of containing one or more antibodies specific for one or more Streptococcus pyogenes antigen;
ii) contacting the biological sample with two or more populations of Streptococcus pyogenes antigen, wherein each of the two or more populations of Streptococcus pyogenes antigen is capable of binding antigen-specific antibodies present in the biological sample to form two or more populations of antigen:antigen-specific antibody complexes if the antigen-specific antibodies are present in the biological sample; and
iii) detecting the complexes, wherein an increase in detection of one or more complexes above a threshold value is indicative of an increased likelihood of developing rheumatic fever or APSGN, or is indicative of a recent exposure to Streptococcus pyogenes in the subject as one criteria for the presence of rheumatic fever or PSGN in the subject;
iv) assessing one or more diagnostic criteria for rheumatic fever or PSGN;
v) and wherein an increase in detection of one or more complexes above a threshold value in conjunction with one or more other diagnostic criteria for rheumatic fever or APSGN is indicative of rheumatic fever or PSGN in the subject.

In another aspect, the present invention relates to a method for detecting the presence of Streptococcus pyogenes infection in a subject, the method comprising:
i) providing a biological sample from a subject that is capable of or suspected of containing one or more antibodies specific for one or more Streptococcus pyogenes antigen;
ii) contacting the biological sample with two or more populations of Streptococcus pyogenes antigen, wherein each of the two or more populations of Streptococcus pyogenes antigen binds antigen-specific antibodies in the biological sample to form two or more populations of antigen:antigen-specific antibody complexes if the antigen-specific antibodies are present in the biological sample; and
iii) detecting the complexes, wherein an increase in detection of one or more complexes indicates the presence of Streptococcus pyogenes in the subject or a recent exposure of the subject to Streptococcus pyogenes.

In another aspect, the present invention relates to a method for detecting Streptococcus pyogenes antigen-specific antibodies in a biological sample, wherein the Streptococcus pyogenes antigen-specific antibodies specifically bind to a Streptococcus pyogenes antigen, the method comprising:
i) providing a biological sample from a subject that is capable of or suspected of containing one or more antibodies specific for one or more Streptococcus pyogenes antigen;
ii) contacting the biological sample with two or more populations of Streptococcus pyogenes antigen, wherein each of the two or more populations of Streptococcus pyogenes antigen binds antigen-specific antibodies in the biological sample to form two or more populations of antigen:antigen-specific antibody complexes if the antigen-specific antibodies are present in the biological sample; and
iii) detecting the complexes, wherein an increase in detection of one or more complexes indicates the biological sample contains antibodies specific for the Streptococcus pyogenes antigen.

In various embodiments, an increase in detection of one or more complexes is detecting the presence of one or more complexes.

Accordingly, in one embodiment, the method for detecting or diagnosing rheumatic fever or poststreptococcal glomerulonephritis (PSGN) including acute poststreptococcal glomerulonephritis (APSGN) in a subject, or an increased likelihood of developing rheumatic fever or PSGN in a subject, comprises:
i) providing a biological sample from the subject that is capable of or suspected of containing one or more antibodies specific for one or more Streptococcus pyogenes antigen;
ii) contacting the biological sample with two or more populations of Streptococcus pyogenes antigen, wherein each of the two or more populations of Streptococcus pyogenes antigen is capable of binding antigen-specific antibodies present in the biological sample to form two or more populations of antigen:antigen-specific antibody complexes if the antigen-specific antibodies are present in the biological sample; and
iii) detecting the complexes, wherein the presence of one or more antigen:antigen-specific antibody complexes is indicative of an increased likelihood of developing rheumatic fever or APSGN, or is indicative of a recent exposure to Streptococcus pyogenes in the subject as one criteria for the presence of rheumatic fever or PSGN in the subject;
iv) assessing one or more diagnostic criteria for rheumatic fever or PSGN;
v) and wherein the presence of one or more antigen:antigen-specific antibody complexes in conjunction with one or more other diagnostic criteria for rheumatic fever or APSGN is indicative of rheumatic fever or PSGN in the subject.

In another embodiment, the method for detecting the presence of Streptococcus pyogenes infection in a subject comprises:
i) providing a biological sample from a subject that is capable of or suspected of containing one or more antibodies specific for one or more Streptococcus pyogenes antigen;
ii) contacting the biological sample with two or more populations of Streptococcus pyogenes antigen, wherein each of the two or more populations of Streptococcus pyogenes antigen binds antigen-specific antibodies in the biological sample to form two or more populations of antigen:antigen-specific antibody complexes if the antigen-specific antibodies are present in the biological sample; and
iii) detecting the complexes, wherein the presence of one or more antigen:antigen-specific antibody complexes indicates the presence of Streptococcus pyogenes in the subject or a recent exposure of the subject to Streptococcus pyogenes.

In another embodiment, the method for detecting Streptococcus pyogenes antigen-specific antibodies in a biological sample, wherein the Streptococcus pyogenes antigen-specific antibodies specifically bind to a Streptococcus pyogenes antigen comprises:
i) providing a biological sample from a subject that is capable of or suspected of containing one or more antibodies specific for one or more Streptococcus pyogenes antigen;
ii) contacting the biological sample with two or more populations of Streptococcus pyogenes antigen, wherein each of the two or more populations of Streptococcus pyogenes antigen binds antigen-specific antibodies in the biological sample to form two or more populations of antigen:antigen-specific antibody complexes if the antigen-specific antibodies are present in the biological sample; and
iii) detecting the complexes, wherein the presence of one or more antigen:antigen-specific antibody complexes indicates the biological sample contains antibodies specific for the Streptococcus pyogenes antigen.

In one aspect, the invention relates to a method of treating a patient suffering from rheumatic fever or PSGN, the method comprising the steps of:
i) providing a biological sample from the subject that is capable of or suspected of containing one or more antibodies specific for Streptococcus pyogenes; and
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) assessing one or more other diagnostic criteria for rheumatic fever or PSGN in the subject;
iv) wherein the presence of Streptococcus pyogenes SpnA antigen-specific complexes, or detection of an amount of Streptococcus pyogenes SpnA antigen-specific complexes above a threshold value, is indicative of a recent exposure to Streptococcus pyogenes in the subject;
v) and wherein the presence of one or more other diagnostic criteria for rheumatic fever or PSGN, together with the absence of Streptococcus pyogenes SpnA antigen-specific complexes, or the detection of an amount of Streptococcus pyogenes SpnA antigen-specific complexes below a threshold value, is indicative of a prior exposure to Streptococcus pyogenes in the subject; and
vi) if the subject has had a recent exposure to Streptococcus pyogenes, then administering treatment for recent-onset rheumatic fever or acute PSGN; and if the subject has had a prior exposure to Streptococcus pyogenes, then administering treatment for established or subsequent Streptococcus pyogenes infection, or administering treatment for rheumatic fever or PSGN.

In one aspect, the invention relates to a method of treating a patient suffering from rheumatic fever or PSGN with an antibiotic effective against Streptococcus pyogenes, the method comprising the steps of:
i) providing a biological sample from the subject that is capable of or suspected of containing one or more antibodies specific for Streptococcus pyogenes; and
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) assessing one or more other diagnostic criteria for rheumatic fever or PSGN in the subject;
iv) wherein the presence of Streptococcus pyogenes SpnA antigen-specific complexes, or detection of an amount of Streptococcus pyogenes SpnA antigen-specific complexes above a threshold value, is indicative of a recent exposure to Streptococcus pyogenes in the subject;
v) and wherein the presence of one or more other diagnostic criteria for rheumatic fever or PSGN, together with the absence of Streptococcus pyogenes SpnA antigen-specific complexes, or the detection of an amount of Streptococcus pyogenes SpnA antigen-specific complexes below a threshold value, is indicative of a prior exposure to Streptococcus pyogenes in the subject; and
vi) if the subject has had a recent exposure to Streptococcus pyogenes, then administering an antibiotic effective against acute or current Streptococcus pyogenes infection; and if the subject has had an earlier (prior) exposure to Streptococcus pyogenes, then administering an antibiotic effective against established or subsequent Streptococcus pyogenes infection.

In one aspect, the invention relates to a method of treating a patient suffering from rheumatic fever or PSGN with an antibiotic effective against Streptococcus pyogenes, the method comprising the steps of:
i) providing a biological sample from the subject that is capable of or suspected of containing one or more antibodies specific for Streptococcus pyogenes;
ii) contacting the biological sample with one or more populations of an antigen from Streptococcus pyogenes SpnA and one or more populations of an antigen from Streptococcus pyogenes DNaseB and/or one or more populations of an antigen from Streptococcus pyogenes SLO, wherein the one or more populations of Streptococcus pyogenes antigen is capable of binding antigen-specific antibodies present in the biological sample to form one or more populations of antigen:antigen-specific antibody complexes if the antigen-specific antibodies are present in the biological sample; and
iii) detecting the complexes, wherein
   a. the presence of Streptococcus pyogenes SpnA-specific complexes, or the detection of an amount of Streptococcus pyogenes SpnA-specific complexes above a threshold value, is indicative of a recent exposure to Streptococcus pyogenes in the subject, and
   b. the presence of Streptococcus pyogenes DNaseB-specific complexes and/or of Streptococcus pyogenes SLO-specific complexes, and the absence of Streptococcus pyogenes SpnA-specific complexes or the detection of an amount of Streptococcus pyogenes SpnA-specific complexes below a threshold value, is indicative of a prior exposure to Streptococcus pyogenes in the subject,
iv) if the subject has had a recent exposure to Streptococcus pyogenes, then administering an antibiotic effective against acute Streptococcus pyogenes infection; and if the subject has had a prior exposure to Streptococcus pyogenes, then administering an antibiotic effective against established or subsequent Streptococcus pyogenes infection.

In one aspect, the invention relates to a method of treating a patient suffering from rheumatic fever or PSGN, the method comprising the steps of:
i) providing a biological sample from the subject that is capable of or suspected of containing one or more antibodies specific for Streptococcus pyogenes;
ii) contacting the biological sample with one or more populations of an antigen from Streptococcus pyogenes SpnA and one or more populations of an antigen from Streptococcus pyogenes DNaseB and/or one or more populations of an antigen from Streptococcus pyogenes SLO, wherein the one or more populations of Streptococcus pyogenes antigen is capable of binding antigen-specific antibodies present in the biological sample to form one or more populations of antigen:antigen-specific antibody complexes if the antigen-specific antibodies are present in the biological sample; and
iii) detecting the complexes, wherein
   a. the presence of Streptococcus pyogenes SpnA-specific complexes, or the detection of an amount of Streptococcus pyogenes SpnA-specific complexes above a threshold value, is indicative of a recent exposure to Streptococcus pyogenes in the subject, and
   b. the presence of Streptococcus pyogenes DNaseB-specific complexes and/or of Streptococcus pyogenes SLO-specific complexes, and the absence of Streptococcus pyogenes SpnA-specific complexes or the detection of an amount of Streptococcus pyogenes SpnA-specific complexes below a threshold value, is indicative of a prior exposure to Streptococcus pyogenes in the subject,
iv) if the subject has had a recent exposure to Streptococcus pyogenes, then administering treatment for recent-onset rheumatic fever or acute PSGN; and if the subject has had a prior exposure to Streptococcus pyogenes, then administering treatment for rheumatic fever or PSGN.

In another aspect, the invention relates to a method of treating rheumatic fever or PSGN in a subject in need thereof, the method comprising the steps of:
i) providing a biological sample from the subject that is capable of or suspected of containing one or more antibodies specific for Streptococcus pyogenes; and
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) assessing one or more other diagnostic criteria for rheumatic fever or PSGN in the subject;
iv) wherein the presence of Streptococcus pyogenes SpnA antigen-specific complexes, or detection of an amount of Streptococcus pyogenes SpnA antigen-specific complexes above a threshold value, is indicative of a recent exposure to Streptococcus pyogenes in the subject;
v) and wherein the presence of one or more other diagnostic criteria for rheumatic fever or PSGN, together with the absence of Streptococcus pyogenes SpnA antigen-specific complexes, or the detection of an amount of Streptococcus pyogenes SpnA antigen-specific complexes below a threshold value, is indicative of a prior exposure to Streptococcus pyogenes in the subject; and
vi) if the subject has had a recent exposure to Streptococcus pyogenes, then administering treatment for recent-onset rheumatic fever or acute PSGN; and if the subject has had a prior exposure to Streptococcus pyogenes, then administering treatment for rheumatic fever or PSGN.

In another aspect, the invention relates to a method of treating rheumatic fever or PSGN in a subject in need thereof, the method comprising the steps of:
i) determining the presence, absence or amount of one or more antibodies specific for Streptococcus pyogenes SpnA in a biological sample from the subject; and
ii) optionally assessing one or more other diagnostic criteria for rheumatic fever or PSGN in the subject;
iii) predicting the subject as suffering from recent-onset rheumatic fever or acute PSGN if the sample comprises one or more antibodies specific for Streptococcus pyogenes SpnA in an amount above a threshold value; and
iv) administering to that subject a therapeutically effective amount of an antibiotic effective against recent Streptococcus pyogenes infection.

In one embodiment, the method of treating rheumatic fever or PSGN in a subject in need thereof comprises the steps of:
i) determining the presence, absence or amount of one or more antibodies specific for Streptococcus pyogenes SpnA in a biological sample from the subject; and
ii) assessing one or more other diagnostic criteria for rheumatic fever or PSGN in the subject;
iii) predicting the subject as suffering from recent-onset rheumatic fever or acute PSGN if the sample comprises one or more antibodies specific for Streptococcus pyogenes SpnA in an amount above a threshold value and the subject has one or more other diagnostic criteria for rheumatic fever or PSGN; and
iv) administering to that subject a therapeutically effective amount of an antibiotic effective against acute Streptococcus pyogenes infection.

In another aspect, the invention relates to a method of treating rheumatic fever or PSGN in a subject in need thereof, the method comprising the steps of:
i) determining the presence, absence or amount of one or more antibodies specific for Streptococcus pyogenes SpnA in a biological sample from the subject; and
ii) assessing one or more other diagnostic criteria for rheumatic fever or PSGN in the subject;
iii) predicting the subject as suffering from rheumatic fever or PSGN if the sample comprises one or more antibodies specific for Streptococcus pyogenes SpnA in an amount below a threshold value and the subject has one or more other diagnostic criteria for rheumatic fever or PSGN; and
iv) administering to the subject a therapeutically effective amount of an antibiotic effective against established or subsequent Streptococcus pyogenes infection.

In various embodiments, the presence, absence or amount of one or more antibodies specific for Streptococcus pyogenes SpnA is determined by contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and

In various embodiments, the amount of one or more antibodies specific for Streptococcus pyogenes SpnA above a threshold value is an antibody titre associated with or indicative of a recent exposure to Streptococcus pyogenes in the subject.

In various embodiments, the amount of one or more antibodies specific for Streptococcus pyogenes SpnA below a threshold value is an antibody titre associated with or indicative of a prior exposure to Streptococcus pyogenes in the subject.

In various embodiments, the threshold value is the amount of SpnA specific antibodies that separates the range of antibody titres or mean antibody titre observed in the population of rheumatic fever or PSGN sufferers within 20 days of their hospitalisation from the range of antibody titres or mean antibody titre observed in the population of rheumatic fever or PSGN sufferers after 20 days of their hospitalisation.

In one embodiment, the reference level, reference threshold, or threshold value is the upper limit of normal (ULN), being the 80^{th} centile of a matched healthy population.

In one aspect, the invention relates to a method for treating a patient with an antibiotic effective against Streptococcus pyogenes, wherein the patient is suffering from or has been exposed to an infection with Streptococcus pyogenes, the method comprising the steps of:
i) providing a biological sample from the subject that is capable of or suspected of containing one or more antibodies specific for Streptococcus pyogenes; and
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) assessing one or more other diagnostic criteria for the presence of Streptococcus pyogenes in the subject or for rheumatic fever or PSGN in the subject;
iv) wherein the presence of Streptococcus pyogenes SpnA antigen-specific complexes, or detection of an amount of Streptococcus pyogenes SpnA antigen-specific complexes above a threshold value, is indicative of a recent exposure to Streptococcus pyogenes in the subject;
v) and wherein the presence of one or more other diagnostic criteria for rheumatic fever or PSGN, together with the absence of Streptococcus pyogenes SpnA antigen-specific complexes or the detection of an amount of Streptococcus pyogenes SpnA antigen-specific complexes below a threshold value, is indicative of a prior exposure to Streptococcus pyogenes in the subject; and
vi) if the subject has had a recent exposure to Streptococcus pyogenes, then administering an antibiotic effective against acute Streptococcus pyogenes infection; and if the subject has had a prior exposure to Streptococcus pyogenes, then administering an antibiotic effective against established or subsequent Streptococcus pyogenes infection.

In one embodiment, the method for treating a patient with an antibiotic effective against Streptococcus pyogenes comprises the steps of:
i) providing a biological sample from the subject that is capable of or suspected of containing one or more antibodies specific for Streptococcus pyogenes;
ii) contacting the biological sample with one or more populations of an antigen from Streptococcus pyogenes SpnA and one or more populations of an antigen from Streptococcus pyogenes DNaseB and/or one or more populations of an antigen from Streptococcus pyogenes SLO, wherein the one or more populations of Streptococcus pyogenes antigen is capable of binding antigen-specific antibodies present in the biological sample to form one or more populations of antigen:antigen-specific antibody complexes if the antigen-specific antibodies are present in the biological sample; and
iii) detecting the complexes, wherein
   a. the presence of Streptococcus pyogenes SpnA-specific complexes, or the detection of an amount of Streptococcus pyogenes SpnA-specific complexes above a threshold value, is indicative of a recent exposure to Streptococcus pyogenes in the subject, and
   b. the presence of Streptococcus pyogenes DNaseB-specific complexes and/or of Streptococcus pyogenes SLO-specific complexes, and the absence of Streptococcus pyogenes SpnA-specific complexes or the detection of an amount of Streptococcus pyogenes SpnA-specific complexes below a threshold value, is indicative of a prior exposure to Streptococcus pyogenes in the subject,
iv) if the subject has had a recent exposure to Streptococcus pyogenes, then administering an antibiotic effective against acute Streptococcus pyogenes infection; and if the subject has had a prior exposure to Streptococcus pyogenes, then administering an antibiotic effective against established or subsequent Streptococcus pyogenes infection.

In various embodiments, treatment for recent-onset rheumatic fever or acute PSGN, or treatment for acute or current Streptococcus pyogenes infection, is administration of an antibiotic effective against acute Streptococcus pyogenes infection, for example administration according to a treatment regimen. For example, a dosage regimen effective against acute Streptococcus pyogenes infection, or dosage regimens effective to treat recent-onset rheumatic fever or acute PSGN, comprises a 10 day course of one or more antibiotics, such as a 10 day course of bicillin. Other suitable treatment regimens will be known to those skilled in the art having the benefit of this disclosure, including certain representative examples disclosed herein.

In various embodiments, treatment for rheumatic fever or PSGN, or treatment for established or subsequent Streptococcus pyogenes infection, is administration of an antibiotic effective against established or subsequent Streptococcus pyogenes infection, for example administration according to a treatment regimen such as a prophylactic treatment regimen. For example, a dosage regimen effective against established or subsequent Streptococcus pyogenes infection, or dosage regimens effective to treat rheumatic fever or PSGN, comprises monthly administration of one or more antibiotics, such as monthly administration of bicillin. Other suitable treatment regimens will be known to those skilled in the art having the benefit of this disclosure, including certain representative examples disclosed herein.

In one example, treatment for rheumatic fever or PSGN, or treatment for established or subsequent Streptococcus pyogenes infection, comprises bed rest and/or hospitalisation.

In one example, administering treatment for recent-onset rheumatic fever or acute PSGN comprises administering to the subject an antibiotic effective against acute Streptococcus pyogenes infection.

In one example, administering treatment for rheumatic fever or PSGN comprises administering to the subject an antibiotic effective against established or subsequent Streptococcus pyogenes infection.

In one example, administering treatment for rheumatic fever or PSGN comprises hospitalising the subject and/or prescribing or consigning the subject to bed rest.

In one aspect, the invention relates to a method of predicting the responsiveness of a patient suffering from rheumatic fever or PSGN to treatment with an antibiotic, the method comprising the steps of:
i) determining the presence, absence or amount of one or more antibodies specific for Streptococcus pyogenes SpnA in a biological sample from the subject; and
ii) assessing one or more other diagnostic criteria for rheumatic fever or PSGN in the subject;
iii) predicting the subject as being likely to be responsive to treatment with an antibiotic if the sample comprises one or more antibodies specific for Streptococcus pyogenes SpnA in an amount below a threshold value and the subject has one or more other diagnostic criteria for rheumatic fever or PSGN; and
iv) administering to the subject a therapeutically effective amount of an antibiotic effective against established or subsequent Streptococcus pyogenes infection.

In one aspect, the invention relates to a method of predicting the responsiveness of a patient suffering from rheumatic fever or PSGN to treatment with an antibiotic, the method comprising the steps of:
i) determining the presence, absence or amount of one or more antibodies specific for Streptococcus pyogenes SpnA in a biological sample from the subject; and
ii) optionally assessing one or more other diagnostic criteria for rheumatic fever or PSGN in the subject;
iii) predicting the subject as being likely to be responsive to treatment with an antibiotic effective for the treatment of recent-onset rheumatic fever or acute PSGN if the sample comprises one or more antibodies specific for Streptococcus pyogenes SpnA in an amount above a threshold value and optionally if the subject has one or more other diagnostic criteria for rheumatic fever or PSGN; and
iv) administering to the subject a therapeutically effective amount of an antibiotic effective against acute Streptococcus pyogenes infection.

In another aspect, the invention relates to a method of determining a treatment regimen for a patient suffering from rheumatic fever or PSGN, the method comprising the steps of:
i) determining the presence, absence or amount of one or more antibodies specific for Streptococcus pyogenes SpnA in a biological sample from the subject; and
ii) assessing one or more other diagnostic criteria for rheumatic fever or PSGN in the subject;
iii) determining the subject should undergo a treatment regimen suitable for treatment of rheumatic fever or PSGN if the sample comprises one or more antibodies specific for Streptococcus pyogenes SpnA in an amount below a threshold value and the subject has one or more other diagnostic criteria for rheumatic fever or PSGN; and
iv) treating the subject in accordance with a treatment regimen suitable for treatment of rheumatic fever or PSGN.

In one embodiment, the treatment of rheumatic fever or PSGN is treatment of chronic rheumatic heart disease.

In one embodiment, the treatment of rheumatic fever or PSGN comprises administering to the subject a therapeutically effective amount of an antibiotic effective against established or subsequent Streptococcus pyogenes infection, for example, a prophylactically-effective amount of such antibiotic.

In one embodiment, the treatment regimen suitable for treatment of rheumatic fever or PSGN is monthly administration of antibiotic, such as monthly administration of bicillin (Benzathine benzylpenicillin).

In another aspect, the invention relates to a method of determining a treatment regimen for a patient suffering from rheumatic fever or PSGN, the method comprising the steps of:
i) determining the presence, absence or amount of one or more antibodies specific for Streptococcus pyogenes SpnA in a biological sample from the subject; and
ii) optionally assessing one or more other diagnostic criteria for rheumatic fever or PSGN in the subject;
iii) determining the subject should undergo a treatment regimen suitable for treatment of recent-onset rheumatic fever or acute PSGN if the sample comprises one or more antibodies specific for Streptococcus pyogenes SpnA in an amount above a threshold value and optionally the subject has one or more other diagnostic criteria for rheumatic fever or PSGN; and
iv) optionally administering to the subject a therapeutically effective amount of an antibiotic effective against acute Streptococcus pyogenes infection in accordance with the treatment regimen.

In one embodiment, if the subject has had a recent exposure to Streptococcus pyogenes, the antibiotic effective against acute Streptococcus pyogenes infection is administered at a dosage rate effective against acute Streptococcus pyogenes infection.

In one embodiment, if the subject has had a recent exposure to Streptococcus pyogenes, the antibiotic effective against acute Streptococcus pyogenes infection is administered at a dosage rate greater than that which is administered to a subject suffering from established or subsequent Streptococcus pyogenes infection or rheumatic fever or PSGN.

In one embodiment, if the subject has had a recent exposure to Streptococcus pyogenes, the antibiotic effective against acute Streptococcus pyogenes infection is administered in conjunction with one or more other therapeutic agents, such as an anti-inflammatory agent, for example aspirin, glucocorticoids such as prednisone, neuroleptic agents such as haloperidol, positive inotropic agents such as digoxin, or in conjunction with one or more other therapies, such as long-term hospitalisation, bed rest, and the like.

In one embodiment, the risk of adverse antibiotic reaction or sequelae from antibiotic administration in a patient exposed to, but not recently exposed to Streptococcus pyogenes, is lower following the administration of an antibiotic effective against chronic Streptococcus pyogenes infection than it would be if the antibiotic administered was an antibiotic administered to a subject suffering from acute Streptococcus pyogenes infection or recent-onset rheumatic fever or acute PSGN.

In one embodiment, the risk of adverse antibiotic reaction or sequelae from antibiotic administration in a patient exposed to, but not recently exposed to Streptococcus pyogenes, is lower following the administration of an antibiotic effective against Streptococcus pyogenes infection in an amount effective against established or chronic Streptococcus pyogenes infection than it would be if that antibiotic were administered in an amount effective to treat acute Streptococcus pyogenes infection or recent-onset rheumatic fever or acute PSGN.

In one embodiment, if the subject has had a recent exposure to Streptococcus pyogenes, then administering an antibiotic effective against acute Streptococcus pyogenes infection comprises parenteral administration of the antibiotic.

In various embodiments, the antibiotic effective against Streptococcus pyogenes is selected from the group comprising Penicillin, Amoxicillin, Oxacillin, Erythromycin, Azithromycin, Clarithromycin, Cephalothin, Cefoxitin, Cefixime, Cefuroxime, Cefotaxime, Ceftriaxone, Vancomycin, Clindamycin, Rifampicin, Ciprofloxacin, Tetracycline, Cotrimoxazole, and Chloramphenicol.

In various embodiments, the antibiotic effective against Streptococcus pyogenes is selected from the group comprising β-lactamins, such as Penicillin, Amoxillin (Amoxicillin), Cefixime, Cefpodoxine, Cefotaxime, Ceftriaxone, Oxacillin; Macrolides, such as Erythromycin, Spiramycine, Azythromycin; Lincosamines, such as Clindamycin; Streptogramines, such as Pristinamycin; Ketolides, such as Telithromycin; Phenicols, such as Chloramphenicol; Glycopeptides, such as Teicoplanin, Vancomycine: Fluoroquinolones, such as Levofloxacin; and Tetracyclines, such as Tetracycline.

In one embodiment, the antibiotic effective against acute Streptococcus pyogenes infection is Penicillin, for example, Penicillin G, including Penicillin G procaine (e.g., Crysticillin) and Penicillin G benzathine (e.g., Bicillin, Bicillin L-A), Penicillin VK (e.g., Beepen-VK, Betapen-VK, Robicillin VK, Veetids), Erythromycin, for example E-Mycin, Ery-Tab, Erythrocin, or Sulfadiazine, for example, Microsulfon.

In one embodiment, if the patient has had a recent exposure to Streptococcus pyogenes, then administering an antibiotic effective against acute Streptococcus pyogenes infection comprises parenteral administration of Penicillin G, Erythromycin, or Sulfadiazine, for example intravenous or intramuscular administration of Penicillin G, Erythromycin, or Sulfadiazine. In another embodiment, oral administration is substituted for parenteral administration.

In various embodiments, the administration of an antibiotic effective against acute Streptococcus pyogenes infection is in accordance with a dosage regimen. In various examples, the dosage regimen comprises administration of a loading dose of said antibiotic, for example over an acute treatment period.

In certain embodiments, the acute treatment period is from about 5 days to about 20 days, for example, from about 8 days to about 15 days, or from about 10 days to about 12 days, including about 10 days.

In one example, a dosage regimen effective against acute Streptococcus pyogenes infection, or dosage regimens effective to treat recent-onset rheumatic fever or acute PSGN, comprises a 10 day course of one or more antibiotics, such as a 10 day course of bicillin. For example, certain exemplary dosage regimens effective against acute Streptococcus pyogenes infection, or dosage regimens effective to treat recent-onset rheumatic fever or acute PSGN, include
i. for Benzathine benzylpenicillin (bicillin): about 900 mg as a single dose, typically by deep IM, for adults and children over 30 kg, and about 450-675 mg as a single dose for children under 30 kg, or 600,000 IU as a single dose IM for patients under 20 kg, and 1.2×10⁶ IU as a single dose IM for patients over 20 kg; all typically for a 10 day period
ii. for Phenoxymethylpenicillin: from 125-250 mg twice daily, orally if IM not possible, typically for 10 days, or 10 mg/kg up to 500 mg twice daily for 10 days;
iii. for Erythromycin: 10 mg/kg up to a maximum of 500 mg, twice daily for 10 days;
iv. for Erythromycin ethyl succinate: 40 mg/kg/day in 2-4 divided doses up to a maximum of 1 g/day for children.

In various embodiments, the administration of an antibiotic effective against established or chronic Streptococcus pyogenes infection is in accordance with a dosage regimen. For example, certain exemplary dosage regimens effective against established or subsequent Streptococcus pyogenes infection, or dosage regimens effective to treat rheumatic fever or PSGN, including chronic rheumatic heart disease, include
i. for Benzathine benzylpenicillin: about 900 mg, typically by deep IM, for adults and children over 30 kg every 4 weeks, and about 450-675 mg for children under 30 kg every 4 weeks, or 600,000 IU intramuscularly every 4 weeks for patients under 20 kg, and 1.2×10⁶ IU intramuscularly every 4 weeks for patients over 20 kg;
ii. for Phenoxymethylpenicillin: from 125-250 mg twice daily, orally if IM not possible, typically for 10 days;
iii. for Erythromycin: 250 mg, twice daily;
iv. for Erythromycin ethyl succinate: 400 mg twice daily.

In another aspect, the invention relates to a method for detecting a recent exposure to Streptococcus pyogenes in a subject, the method comprising:
i) providing a biological sample from the subject that is capable of or suspected of containing one or more antibodies specific for Streptococcus pyogenes;
ii) contacting the biological sample with one or more populations of an antigen from Streptococcus pyogenes SpnA, wherein the one or more populations of Streptococcus pyogenes SpnA antigen is capable of binding SpnA antigen-specific antibodies present in the biological sample to form one or more populations of SpnA antigen:SpnA antigen-specific antibody complexes if the SpnA antigen-specific antibodies are present in the biological sample; and
iii) detecting the presence or absence of the complexes,
iv) optionally assessing one or more other diagnostic criteria for the presence of Streptococcus pyogenes in or rheumatic fever or PSGN for the subject;
v) wherein the presence of Streptococcus pyogenes SpnA antigen-specific complexes, or an amount of Streptococcus pyogenes SpnA antigen-specific complexes above a threshold value, is indicative of a recent exposure to Streptococcus pyogenes in the subject;
vi) and wherein the presence of one or more other diagnostic criteria for rheumatic fever or PSGN together with the absence of Streptococcus pyogenes SpnA antigen-specific complexes, or an amount of Streptococcus pyogenes SpnA antigen-specific complexes below a threshold value, is indicative of a prior exposure to Streptococcus pyogenes in the subject.

In another aspect, the invention relates to a method for detecting or diagnosing rheumatic fever or poststreptococcal glomerulonephritis (PSGN) including acute poststreptococcal glomerulonephritis (APSGN) in a subject, or an increased likelihood of developing rheumatic fever or PSGN in a subject, the method comprising:
i) providing a biological sample from the subject that is capable of or suspected of containing one or more antibodies specific for Streptococcus pyogenes;
ii) contacting the biological sample with one or more populations of an antigen from Streptococcus pyogenes SpnA, wherein the one or more populations of Streptococcus pyogenes SpnA antigen is capable of binding SpnA antigen-specific antibodies present in the biological sample to form one or more populations of SpnA antigen:SpnA antigen-specific antibody complexes if the antigen-specific antibodies are present in the biological sample; and
iii) detecting the presence or absence of the complexes;
iv) assessing one or more other diagnostic criteria for rheumatic fever or PSGN for the subject;
v) wherein the presence of one or more other diagnostic criteria for rheumatic fever or PSGN together with the presence of Streptococcus pyogenes SpnA-specific complexes, or detection of an amount of Streptococcus pyogenes SpnA-specific complexes above a threshold value, is indicative of an increased likelihood of developing rheumatic fever or APSGN, or is indicative of a recent exposure to Streptococcus pyogenes in the subject as one criteria for the presence of rheumatic fever or PSGN in the subject;
vi) and wherein the presence of one or more other diagnostic criteria for rheumatic fever or PSGN together with the absence of Streptococcus pyogenes SpnA-specific complexes, or an amount of Streptococcus pyogenes SpnA-specific complexes below a threshold value, is indicative of rheumatic fever or PSGN in the subject, or is indicative of a prior exposure to Streptococcus pyogenes in the subject as one criteria for the presence of rheumatic fever or PSGN in the subject, such as increased risk of or from subsequent Streptococcus pyogenes infection.

In one embodiment, the one or more other diagnostic criteria is the presence or absence of antibodies specific for one or more Streptococcus pyogenes antigens other than SpnA. For example, the one or more other diagnostic criteria is the presence or absence of antibodies specific for Streptococcus pyogenes DNaseB, or antibodies specific for Streptococcus pyogenes SLO.

Any of the embodiments disclosed herein may relate to any of the aspects set out herein. To avoid any confusion, it will be evident from the disclosure herein that any of the aspects disclosed herein, for example any of the methods described herein, will in certain embodiments employ one or more Streptococcus pyogenes nuclease A (SpnA) polypeptides, such as one or more truncated SpnA polypeptides, or one or more fragments of SpnA, as herein described. For example, in various embodiments the one or more antigens or one or more populations of an antigen from Streptococcus pyogenes SpnA are present as one or more SpnA polypeptides, such as one or more truncated SpnA polypeptides or one or more SpnA fragments, as herein described.

Similarly, when used in any of the aspects disclosed herein, for example any of the methods described herein, the one or more Streptococcus pyogenes DNaseB antigens or polypeptides will in certain embodiments be one or more DNaseB polypeptides, such as one or more antigenic fragments of DNaseB, as disclosed herein. For example, in various embodiments the one or more antigens or one or more populations of an antigen from Streptococcus pyogenes DNaseB are present as one or more DNaseB polypeptides, such as one or more DNaseB fragments, as herein described.

Likewise, when used in any of the aspects disclosed herein, for example any of the methods described herein, the one or more Streptococcus pyogenes SLO antigens or SLO polypeptides will in certain embodiments be one or more DNaseB polypeptides, such as one or more antigenic fragments of SLO, as disclosed herein. For example, in various embodiments the one or more antigens or one or more populations of an antigen from Streptococcus pyogenes SLO are present as one or more SLO polypeptides, such as one or more SLO fragments, as herein described.

In one embodiment, the presence of two or more populations of antigen:antigen-specific antibody complexes indicates the presence of Streptococcus pyogenes in the subject, or indicates a recent exposure of the subject to Streptococcus pyogenes, or indicates the biological sample contains antibodies specific for two or more Streptococcus pyogenes antigens.

In one embodiment, the increase in detection of one or more complexes is an increase relative to a reference level of the antigen established for each test population.

In one embodiment, the one or more antibodies specific for one or more Streptococcus pyogenes antigens is one or more serum antibodies.

In one embodiment, the one or more serum antibodies is one or more IgG antibodies.

In one embodiment, the one or more serum antibodies is one or more IgA antibodies or one or more IgM antibodies.

In one embodiment, the one or more other diagnostic criteria is the presence or absence of one or more clinical symptoms associated with rheumatic fever or PSGN.

In one embodiment, the one or more clinical symptoms are selected from migratory polyarthritis, carditis, hematuria, erythema marginatum, subcutaneous nodules, Seydenham's Chorea, or pyoderma.

In one embodiment, one or more of the Streptococcus pyogenes antigens is an antigen from one of the following proteins:
i) Streptococcus pyogenes nuclease A (SpnA),
ii) Deoxyribonuclease-B (DNaseB), or
iii) Streptolysin-O (SLO).

In one embodiment, one or more of the Streptococcus pyogenes antigens is selected from the group consisting of:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8, or
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5, or
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2, or
x) any combination of two or more of i) to ix) above.

In one embodiment, the biological sample is contacted with a population of each of the following Streptococcus pyogenes antigens:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8,
and
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5,
   and
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2.

In one embodiment, the biological sample is contacted with a population of each of the following Streptococcus pyogenes antigens:
i) Streptococcus pyogenes nuclease A (SpnA),
ii) Deoxyribonuclease-B (DNaseB), and
iii) Streptolysin-O (SLO).

In one embodiment, the biological sample is contacted with a population of each of the following Streptococcus pyogenes antigens:
i) an antigenic fragment of SpnA comprising, consisting essentially of, or consisting of the amino acid sequence of SEQ ID No. 8,
ii) an antigenic fragment of DNaseB comprising, consisting essentially of, or consisting of the amino acid sequence of SEQ ID No. 5, and
iii) an antigenic fragment of SLO comprising, consisting essentially of, or consisting of the amino acid sequence of SEQ ID No. SEQ ID No. 1 or SEQ ID No. 2.

In one embodiment, the two or more populations of Streptococcus pyogenes antigen are present in a composition.

In one embodiment, one or more of the Streptococcus pyogenes antigens is labelled with a detectable label, and/or is coupled to a microparticle, bead, or detectable agent.

In one embodiment, one or more of the populations of Streptococcus pyogenes antigens is covalently bound to beads or to microparticles.

In one embodiment, each of the populations of Streptococcus pyogenes antigens is covalently bound to beads or to microparticles, optionally wherein each of the different populations of beads or microparticles is distinguishable one from the other.

In one embodiment, the beads are polystyrene beads, magnetic beads, carboxylated beads, functionalised beads, or wherein the microparticles are polystyrene microparticles, magnetic microparticles, carboxylated microparticles, or functionalised microparticles. In various examples, the beads are suitable for use in multiplexes assays, such as those comprising two or more populations of beads or microparticles, where each population is conjugated to a different antigen. In various examples, the beads or microparticles are suitable for use in immunoassays such as CBA, luminex assays, or the like.

In one embodiment, detecting the antigen:antibody complexes comprises exposing the complexes to a specific binding partner that carries a detectable label and detecting a signal from the label if the antigen-specific antibodies are present in the biological sample.

In one embodiment, the specific binding partner comprises an antibody or fragment thereof.

In one embodiment, the specific binding partner is an anti-IgG antibody, an anti-IgG-PE, or fragment thereof.

In one embodiment, the antigen:antibody complexes are detected using a flow instrument, an immunoassay such as a plate-based immunological assay, electrophoresis and/or immunoblot, an immunochromatographic strip, an electronic biosensor, a resonance biosensor, or a microfluidic device or sensor.

In one embodiment, the immunoassay, such as a plate based immunoassay, is an ELISA or a luminex assay.

In one embodiment, the antigen:antibody complexes are detected in a luminex assay, for example in a luminex assay as herein exemplified.

In one embodiment, the presence of one or more complexes or of one or more of the antigen specific antibodies is detected using a detectably labelled secondary antibody.

In one embodiment, the detectably labelled secondary antibody is anti-IgG-PE.

In one embodiment, the Streptococcus pyogenes antigens are detectably labelled.

In one embodiment, the detectable label is a fluorophore.

In one embodiment, the biological sample is obtained from a mammalian species.

In one embodiment, the biological sample is a bodily fluid sample.

In one embodiment, the subject is a human subject.

In another aspect, the invention relates to an isolated, purified, or recombinant SpnA polypeptide, wherein said SpnA polypeptide is:
i) N-terminally truncated;
ii) C-terminally truncated; or
iii) both N-terminally truncated and C-terminally truncated;
with respect to wild type SpnA.

In one embodiment, the SpnA polypeptide
i) is immunogenic, or
ii) is immunologically cross-reactive with wild type SpnA, or
iii) is detectably labelled, or
iv) has enhanced stability when stored at room temperature compared to wild type SpnA, or
v) comprises 10 or more contiguous amino acids from SEQ ID No. 8, or
vi) is any combination of two or more of i) to v) above.

In one embodiment, the SpnA polypeptide has an elevated mean Tagg compared to wild type SpnA, wherein Tagg is the temperature at which 50% of the proteins are aggregated, for example, as determined by SDS-PAGE analysis. For example, the SpnA polypeptide has a mean Tagg of at least about 50° C., such as a mean Tagg of at least about 50° C. as determined by SDS-PAGE analysis.

In one embodiment, the SpnA polypeptide has a higher degree of thermostability at a temperature of from about 35° C. to about 60° C. compared to wild type SpnA polypeptide.

In one embodiment, the polypeptide has enhanced thermostablity, enhanced immunogenic stability, or both enhanced thermostability and enhanced immunogenic stability.

Again, for the avoidance of doubt, those skilled in the art will appreciate that any of the aspects disclosed herein, for example any of the methods described herein, will in certain embodiments employ one or more truncated Streptococcus pyogenes nuclease A (SpnA) polypeptides, such as one or more truncated SpnA polypeptides described above, including one or more fragments of SpnA as herein described. For example, in various embodiments the one or more antigens or one or more populations of an antigen from Streptococcus pyogenes SpnA are present as one or more truncated SpnA polypeptides, such as one or more N-terminally truncated SpnA polypeptides, or one or more C-terminally truncated SpnA polypeptides, or one or more SpnA fragments, as herein described.

In another aspect, the invention relates to a composition comprising the isolated, purified, or recombinant SpnA polypeptide as described herein.

In a further aspect, the invention relates to a composition comprising detectably labelled SpnA polypeptide, such as a recombinant SpnA polypeptide as herein described.

In one embodiment, the detectably labelled SpnA is a truncated SpnA polypeptide, such as one or more N-terminally truncated SpnA polypeptide, or one or more C-terminally truncated SpnA polypeptide, or one or more SpnA fragments, as herein described.

In another aspect, the invention relates to a bead or microparticle comprising or to which has been bound one or more Streptococcus pyogenes antigens or one or more populations of Streptococcus pyogenes antigens, such as one or more Streptococcus pyogenes nuclease A (SpnA) polypeptides as herein described.

In another aspect, the invention relates to a composition comprising one or more beads or microparticles as herein described.

In still a further aspect, the invention relates to a kit for detecting or diagnosing rheumatic fever or PSGN in a subject, for detecting the presence of Streptococcus pyogenes infection in a subject, or for detecting Streptococcus pyogenes antigen-specific antibodies in a biological sample, the kit comprising a composition comprising at least one of the Streptococcus pyogenes antigens selected from the group consisting of:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8, or
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5, or
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2,
   optionally at least one composition comprising a reference antibody control, wherein the antibody control comprises an antibody specific to one of the Streptococcus pyogenes antigens present in the kit,
   optionally one or more reagents for constituting the medium favourable for contacting the one or more antigens with a biological sample,
   optionally one or more reagents enabling the detection of a complex formed between the one or more antigens and one or more Streptococcus pyogenes antigen specific antibodies present in a biological sample,
   and instructions for use.

In one embodiment, at least one of the Streptococcus pyogenes antigens is covalently bound to a bead or a microparticle.

In one embodiment, the composition comprises a population of:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8.

In one embodiment, the composition comprises a population of each of the following Streptococcus pyogenes antigens:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8,
   and
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5,
   and
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2.

In various embodiments of methods provided herein, the method further comprises providing before step ii) a kit as described herein.

In various embodiments of methods or kits provided herein, one or more of the Streptococcus pyogenes antigens is selected from the group comprising antistreptolysin (ASO), antihyaluronidase (AHase), antistreptokinase (ASKase), antinicotinamide-adenine dinucleotidase (anti-NAD).

In one embodiment, the method comprises the use of or the composition or kit comprising two or more populations of beads or microparticles, wherein each population of beads or microparticles comprises a different Streptococcus pyogenes antigen, and wherein at least one of the populations of beads or microparticles comprises a population of one of the following Streptococcus pyogenes antigens:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8,
and wherein the beads or microparticles are amenable to use in a flow instrument, an immunoassay such as a plate-based immunological assay, electrophoresis and/or immunoblot, an immunochromatographic strip, an electronic biosensor, a resonance biosensor, a microfluidic device or sensor, including for example, in a ELISA, Luminex, or CBA assay.

In various embodiments, the use is in any of the methods as herein described. For example, the use is a use including but not limited to, any one of the following: a method for detecting recent exposure to Streptococcus pyogenes in a subject, a method for detecting or diagnosing rheumatic fever or poststreptococcal glomerulonephritis (PSGN) including acute poststreptococcal glomerulonephritis (APSGN) in a subject, a method for detecting or diagnosing an increased likelihood of developing rheumatic fever or PSGN in a subject, a method for detecting the presence of Streptococcus pyogenes infection in a subject, a method for detecting Streptococcus pyogenes antigen-specific antibodies in a biological sample, a method of treating a patient suffering from rheumatic fever or PSGN, a method of treating a patient suffering from rheumatic fever or PSGN with an antibiotic effective against Streptococcus pyogenes, a method for treating a patient with an antibiotic effective against Streptococcus pyogenes, wherein the patient is suffering from or has been exposed to an infection with Streptococcus pyogenes, a method of predicting the responsiveness of a patient suffering from rheumatic fever or PSGN to treatment with an antibiotic, a method of determining a treatment regimen for a patient suffering from rheumatic fever or PSGN, or a method for detecting a recent exposure to Streptococcus pyogenes in a subject.

Optionally, one or more of the two or more populations of beads or microparticles comprises a population of one of the following Streptococcus pyogenes antigens:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8, or
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5, or
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2.

In another aspect, the invention relates to an isolated, purified, or recombinant SpnA polypeptide, a bead or microparticle comprising or to which has been bound one or more Streptococcus pyogenes antigens or one or more populations of Streptococcus pyogenes antigens, such as one or more Streptococcus pyogenes nuclease A (SpnA) polypeptides, a composition comprising the isolated, purified, or recombinant SpnA polypeptide as described herein, a composition comprising detectably labelled SpnA, a composition comprising one or more beads of microparticles as herein described, a kit for detecting or diagnosing rheumatic fever or PSGN in a subject, a kit for detecting the presence of Streptococcus pyogenes infection in a subject, or a kit for detecting Streptococcus pyogenes antigen-specific antibodies in a biological sample, for any one of the following: for detecting recent exposure to Streptococcus pyogenes in a subject; for detecting or diagnosing rheumatic fever or poststreptococcal glomerulonephritis (PSGN) including acute poststreptococcal glomerulonephritis (APSGN) in a subject; for detecting or diagnosing an increased likelihood of developing rheumatic fever or PSGN in a subject; for detecting the presence of Streptococcus pyogenes infection in a subject; for detecting Streptococcus pyogenes antigen-specific antibodies in a biological sample; for treating a patient suffering from rheumatic fever or PSGN; for treating a patient suffering from rheumatic fever or PSGN with an antibiotic effective against Streptococcus pyogenes; for treating a patient with an antibiotic effective against Streptococcus pyogenes, wherein the patient is suffering from or has been exposed to an infection with Streptococcus pyogenes; for predicting the responsiveness of a patient suffering from rheumatic fever or PSGN to treatment with an antibiotic; for determining a treatment regimen for a patient suffering from rheumatic fever or PSGN; or for detecting a recent exposure to Streptococcus pyogenes in a subject.

In various embodiments, the polypeptide, bead or microparticle, composition, or kit, is or comprises at least one of the Streptococcus pyogenes antigens selected from the group consisting of:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8, or
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5, or
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2.

In various embodiments, the subject or patient is a subject having an amount of or an antibody titre of anti-Streptococcus pyogenes SpnA antibodies above a threshold value, wherein an amount or antibody titre of said antibodies above said threshold value is indicative of a recent exposure to Streptococcus pyogenes. For example, the threshold value is the upper limit of normal (ULN), being the 80^{th} centile of a matched healthy population.

In other embodiments, the subject or patient is a subject having an amount of or antibody titre of anti-Streptococcus pyogenes SpnA antibodies below a threshold value, wherein an amount or antibody titre of said antibodies below said threshold value is indicative of a prior exposure to Streptococcus pyogenes. For example, the threshold value is the upper limit of normal (ULN), being the 80^{th} centile of a matched healthy population.

In another aspect, the invention relates to a method for detecting recent exposure to Streptococcus pyogenes in a subject, the method comprising:
i) providing a biological sample from the subject that is capable of or suspected of containing one or more antibodies specific for Streptococcus pyogenes SpnA;
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) detecting the complexes, wherein an increase in detection of one or more complexes above a threshold value is indicative of a recent exposure to Streptococcus pyogenes in the subject.

In another aspect, the invention relates to a method for detecting or diagnosing rheumatic fever or poststreptococcal glomerulonephritis (PSGN) including acute poststreptococcal glomerulonephritis (APSGN) in a subject, or an increased likelihood of developing rheumatic fever or PSGN in a subject, the method comprising:
i) providing a biological sample from the subject that is capable of or suspected of containing one or more antibodies specific for Streptococcus pyogenes SpnA;
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) detecting the complexes, wherein an increase in detection of one or more complexes above a threshold value is indicative of an increased likelihood of developing rheumatic fever or APSGN, or is indicative of a recent exposure to Streptococcus pyogenes in the subject as one criteria for the presence of rheumatic fever or PSGN in the subject;
iv) assessing one or more diagnostic criteria for rheumatic fever or PSGN for the subject;
v) and wherein an increase in detection of one or more complexes above a threshold value in conjunction with one or more other diagnostic criteria for rheumatic fever or APSGN is indicative of rheumatic fever or APSGN in the subject.

In one embodiment, the one or more diagnostic criteria is the presence or absence of one or more clinical symptoms associated with rheumatic fever or PSGN.

In one embodiment, the one or more clinical symptoms are selected from migratory polyarthritis, carditis, hematuria, erythema marginatum, subcutaneous nodules, Seydenham's Chorea, or pyoderma.

In another aspect, the invention relates to a method for detecting the presence of Streptococcus pyogenes infection in a subject, the method comprising:
i) providing a biological sample from a subject that is capable of or suspected of containing one or more antibodies specific for Streptococcus pyogenes SpnA;
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) detecting the complexes, wherein an increase in detection of one or more complexes indicates the presence of Streptococcus pyogenes in the subject or a recent exposure of the subject to Streptococcus pyogenes.

In another aspect, the invention relates to a method for detecting Streptococcus pyogenes antigen-specific antibodies in a biological sample, wherein the Streptococcus pyogenes antigen-specific antibodies specifically bind to Streptococcus pyogenes SpnA, the method comprising:
i) providing a biological sample from a subject that is capable of or suspected of containing one or more antibodies specific for Streptococcus pyogenes SpnA;
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) detecting the complexes, wherein an increase in detection of one or more complexes indicates the biological sample contains antibodies specific for the Streptococcus pyogenes antigen.

In one embodiment, the poststreptococcal glomerulonephritis is acute poststreptococcal glomerulonephritis (APSGN).

In various embodiments, the increase in detection of one or more complexes is an increase in detection of one or more complexes above a threshold value.

In various embodiments, the reference level, reference threshold, or threshold value (used interchangeably herein and also referred to as the cut-off) is determined for a particular population. In one example, for recent-onset rheumatic fever (ARF) in a given country, for example, for ARF in NZ, the threshold is determined using a cohort of healthy, well matched volunteers. The cut-offs (or reference levels) thus established is then applied to all ARF in NZ. It will be appreciated that reference thresholds may differ across countries, ethnic groups, and populations, and thus in certain embodiments different countries or populations each determine their own reference thresholds. In one embodiment, the reference level, reference threshold, or threshold value is the upper level of normal (ULN), being the 80^{th} centile of a matched healthy population.

In one embodiment, the reference threshold for detecting a Streptococcus pyogenes antigen is the mean titre for that antibody observed in samples obtained from a population of rheumatic fever or PSGN sufferers within 20 days of their hospitalisation. For example, the reference threshold for a Streptococcus pyogenes antigen for use in a method described herein is the mean antigen-specific antibody titre observed using the methods described herein in samples obtained from a population of rheumatic fever or PSGN sufferers demographically comparable to the subject within 20 days of their hospitalisation.

In one embodiment, the reference threshold for detecting anti-SpnA antibody:SpnA complexes, for example the reference threshold for SpnA for use in determining a recent exposure to Streptococcus pyogenes, is the mean anti-SpnA antibody titre observed in samples obtained from a population of rheumatic fever or PSGN sufferers within 20 days of their hospitalisation. For example, the reference threshold for SpnA for use in determining a recent exposure to Streptococcus pyogenes is the mean anti-SpnA antibody titre observed using the methods described herein in samples obtained from a population of rheumatic fever or PSGN sufferers demographically comparable to the subject within 20 days of their hospitalisation.

Other objects, aspects, features and advantages of the present invention will become apparent from the following description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

Further aspects of the present invention will become apparent from the following description which is given by way of example only and with reference to the accompanying drawings in which:

FIG. 1 presents scatter plots showing the correlation between singleplex and multiplex Median Fluorescence Values (MFI) determined by the Cytometric Bead Array for SLO (FIG. 1A), DNaseB (FIG. 1B) and SpnA (FIG. 1C) in 10 serum samples. Linear regression analysis was performed and R² were determined as follows: SLO=0.999; DNaseB B=0.998; and SpnA=0.998;

FIG. 2 shows the results of ELISA for the purified IgG against the three Group A Streptococcus antigens. The antibodies were purified from IVIG using affinity chromatography resulting in IgG with specificity for SLO (A), DNaseB (B) and SpnA (C). The error bars represent standard deviations;

FIG. 3 presents scatter plots showing serum antibody concentrations determined by Cytometric Bead Array for SLO (A), DNaseB (B) and SpnA (C). The ULN values for each antigen are shown (dotted line). Kruskal-Wallis one-way analysis of variance was performed to determine p-values;

FIG. 4 presents scatter plots showing the correlation between commercially available tests and the Cytometric Bead Array for SLO (A), DNaseB (B). Linear regression analysis was performed and R² were determined as SLO=0.968 and DNaseB B=0.934;

FIG. 5 presents standard curves for SLO, DNAse B and SpnA for Cytometric Bead Array fitted with a five-parameter logistic formula on FCAP Array software. Purified IgG specific to SLO (500 ng/ml), DNAse B (500 ng/ml) and SpnA (1500 ng/ml) were diluted two-fold and incubated with the antigen-coupled beads;

FIG. 6 depicts the amino acid sequence of a recombinant fragment of SLO comprising amino acids 34-571 (FIG. 6A), while the amino acid sequence of a detoxified SLO analogue is presented in FIG. 6B. The substituted amino acids are highlighted and underlined;

FIG. 7 depicts the amino acid sequence of a recombinant fragment of DNaseB, comprising amino acids 43-271;

FIG. 8 presents the amino acid sequence of a recombinant fragment of SpnA, comprising amino acids 28-854;

FIG. 9 presents three scatter plots comparing singleplex luminex assays (each antigen individually) with multiplex luminex assays in which the three antigen beads were mixed in equal parts and incubated with the test sera in a single assay. FIG. 9A presents the correlation between the MFI for SLO in the singleplex luminex assay and in the multiplex luminex assay; FIG. 9B presents the correlation between the MFI for DNaseB in the singleplex luminex assay and in the multiplex luminex assay; and FIG. 9C presents the correlation between the MFI for SpnA in the singleplex luminex assay and in the multiplex luminex assay; as described herein in Example Two;

FIG. 10 presents two scatter plots showing the correlation between commercially available tests and the luminex assay for SLO (FIG. 10A), and for DNaseB (FIG. 10B). Linear regression analysis was performed and R² were determined as SLO=0.933 and DNaseB B=0.942;

FIG. 11 presents three graphs showing the levels of IgG antibodies present in sera collected from patients as determined by luminex assay, where the sera is segregated by days from hospitalization. No significant difference was observed in anti-SLO antibody concentration in sera collected <20 days from hospitalization compared to sera collected >20 days from hospitalization (FIG. 11, left hand panel), nor in anti-DNaseB antibody concentrations between these two groups (FIG. 11, middle panel). In contrast, a significant reduction in anti-SpnA antibody concentration was observed in sera collected >20 days from hospitalization when compared to that in sera collected <20 days from hospitalisation (FIG. 11, right hand panel), as described in Example Two herein.

FIG. 12 presents three graphs showing the levels of IgG antibodies present in sera collected from patients as determined by luminex assay, where the sera are segregated by days from hospitalization. No significant difference was observed in anti-SLO antibody concentration in sera collected <20 days from hospitalization compared to sera collected >20 days from hospitalization (FIG. 12, left hand panel), nor in anti-DNaseB antibody concentrations between these two groups (FIG. 12, middle panel). In contrast, a significant reduction in anti-SpnA antibody concentration was observed in sera collected >20 days from hospitalization when compared to that in sera collected <20 days from hospitalisation (FIG. 12, right hand panel), as described in Example Three herein.

FIG. 13 presents two chromatographs showing an analysis of the thermostability of native SpnA and the truncated SpnA polypeptide disclosed herein, as described in Example Four. FIG. 13A is a chromatograph of an SDS-PAGE analysis of the full length SpnA polypeptide, stored under optimal conditions (0 days) at for five days at room temperature (5 days). FIG. 13B is a chromatograph of an SDS-PAGE analysis of the truncated SpnA polypeptide, again stored under optimal conditions (0 days) at for five days at room temperature (5 days).

FIG. 14 presents one chromatograph and two graphs showing an analysis of the thermostability of native SpnA and the truncated SpnA polypeptide disclosed herein, as described in Example Five. FIG. 14A is a chromatograph of an SDS-PAGE analysis of the percentage of folded protein at each temperature. FIG. 14B is a graph showing the Tagg (temperature at which 50% of proteins are aggregated) for each protein at each temperature. FIG. 14C is a graph of the mean Tagg value for each polypeptide, depicting the higher mean Tagg determined for the truncated construct of 51.0+/−0.6° C., significantly higher than that determined for the native SpnA polypeptide (47.5+/−0.9° C.).

### DETAILED DESCRIPTION

The present invention generally relates to methods and compositions for detecting the presence of GAS-specific antibodies in biological samples. The detection of such antibodies is useful in identifying subjects having increased risk of postinfection immune sequelae, and in identifying subjects who may benefit from particular treatments, in addition to other uses which will become apparent to a person skilled in the art on reading the following disclosure.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

In certain embodiments, the invention relates to a method for assisting in the diagnosis of rheumatic fever or poststreptococcal glomerulonephritis (PSGN) or assessing the propensity for developing rheumatic fever or PSGN in a subject comprising determining the presence or amount of one or more antibodies specific for one or more Streptococcus pyogenes antigens in a biological sample from a subject, wherein an elevated level of said antibodies in the biological sample relative to the level of said antibodies in a control is indicative of rheumatic fever or PSGN or an increased propensity for developing rheumatic fever or PSGN.

In other embodiments, the invention relates to a method for determining the efficacy of a treatment for rheumatic fever or PSGN in a subject comprising determining the presence or amount of one or more antibodies specific for one or more Streptococcus pyogenes antigens from one or more biological samples obtained from the subject before or during the course of the treatment, wherein a decrease in the level of the one or more antibodies specific for one or more Streptococcus pyogenes antigens in samples obtained from the subject over time is indicative that the treatment is efficacious.

In a further embodiment, the invention relates to a method for selecting a subject for treatment of rheumatic fever or PSGN comprising (a) determining the presence or amount of one or more antibodies specific for one or more Streptococcus pyogenes antigens in a biological sample obtained from the subject; (b) comparing the level of said antibodies in the biological sample to the level of said antibodies in a control; and (c) selecting the subject for treatment when the level of said antibodies in the biological sample is higher than the level of said antibodies in the control.

The invention in part relies on the affinity of antibodies for antigenic components—the capability of antibodies to specifically recognize and bind to an antigen or an epitope, and the determination of this specific recognition and binding. In other words, the invention in part relies on the detection or identification of complexes formed when an antibody recognizes and binds to a specific epitope.

The term “affinity” refers to a measure of the strength of the binding of an individual epitope with the complementarity determining region (CDR) of a binding molecule—in the context of the current invention typically an immunoglobulin molecule. The term “avidity” refers to the overall stability of the complex between a population of immunoglobulins and antigen(s), that is, the functional combining strength of an immunoglobulin mixture with the antigen. Those skilled in the art will understand that avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. The affinity or avidity of an antibody for an antigen can be determined experimentally using any suitable method as are well known in the art, including certain methods described herein. General techniques for measuring the affinity of an antibody for an antigen include ELISA, RIA, and surface plasmon resonance. The measured affinity of a particular antibody:antigen interaction can vary if measured under different conditions, e.g., salt concentration, pH, buffer, temperature. Thus, and particularly when comparative data is desired, measurements of affinity and other antigen-binding parameters, e.g., KD, IC50, are typically made with standardized solutions of antibody and antigen, a standardized buffer, and standardized assay conditions.

By “specifically binding”, or “specifically recognizing”, used interchangeably herein, it is generally meant that a binding molecule, e.g., an antibody binds to an epitope via its antigen-binding domain, and that the binding entails some complementarity between the antigen-binding domain and the epitope. Accordingly, an antibody is said to “specifically bind” to an epitope when it binds to that epitope, via its antigen-binding domain more readily than it would bind to a random, unrelated epitope. Such an antibody may be referred to herein as an “antibody specific for” the recited epitope or class of epitopes. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “x” with a higher specificity than it has for related epitope “y”.

A reference to “determining” as used herein includes estimating, quantifying, calculating or otherwise deriving the amount of the referenced material (for example, the antibody, antigen, or biomarker) present in a specific sample. This may be achieved by measuring an end point indication that may be for example, the appearance of a detectable product, any detectable change in for example substrate levels or any change in the rate of the appearance of the product or the disappearance of the substrate, or measuring the amount of antibody bound to an antigen, biomarker, complex, or other reagent, as described herein.

Where present, the term “immunological binding characteristics,” or other binding characteristics of an antibody with an antigen, in its various grammatical forms, refers to the specificity, affinity, cross-reactivity, and other binding characteristics of an antibody.

As used herein, the terms “immunogenic stability”, “immunological stability”, and grammatical equivalents contemplates a maintenance of one or more immunogenic or immunological characteristics, such as a maintenance of an ability to engender a given, including a specific, immunological response, such as the ability to be bound by or recognized by an antibody, or the ability to elicit a cell-mediated immunological response. For example, when used with reference to a particular antigen, polypeptide, or other agent, immunogenic stability or immunological stability includes, for example, the maintenance of antibody-specific recognition, for example the ability of the antigen, polypeptide or other agent to be bound by a specific antibody. It will be understood that immunological stability may also refer to the stability of an antibody, such as the maintained ability to recognize and bind to an epitope.

The term “preferentially binding” as used herein contemplates a binding, for example of an antibody to an epitope, that occurs more readily than to another epitope, such as a related, similar, homologous, or analogous epitope. Thus, an antibody which “preferentially binds” to a given epitope would more likely bind to that epitope than to another epitope, even in circumstances where such an antibody may exhibit some cross-reactivity with the other epitope.

For example, an antibody may be considered to bind a first epitope preferentially if it binds said first epitope with a dissociation constant (K_{D}) that is less than the antibody's K_{D} for the second epitope. In one embodiment, an antibody may be considered to bind a first antigen preferentially if it binds the first epitope with an affinity that is at least one order of magnitude less than the antibody's K_{D} for the second epitope. In another embodiment, an antibody may be considered to bind a first epitope preferentially if it binds the first epitope with an affinity that is at least two orders of magnitude less than the antibody's K_{D} for the second epitope.

In another example, an antibody may be considered to bind a first epitope preferentially if it binds the first epitope with an association rate constant (on rate, (k(on)) that is less than the antibody's k(on) for the second epitope. In one embodiment, an antibody may be considered to bind a first epitope preferentially if it binds the first epitope with a k(on) that is at least one order of magnitude greater than the antibody's k(on) for the second epitope. In another embodiment, an antibody may be considered to bind a first epitope preferentially if it binds the first epitope with a k(on) that is at least two orders of magnitude greater than the antibody's k(on) for the second epitope.

In other examples, an antibody may be considered to bind a first epitope preferentially if it binds the first epitope with a dissociation rate constant (off rate (k(off)) that is less than the antibody's k(off) for the second epitope. In one embodiment, an antibody may be considered to bind a first epitope preferentially if it binds the first epitope with a k(off) that is at least one order of magnitude less than the antibody's k(off) for the second epitope. In another embodiment, an antibody may be considered to bind a first epitope preferentially if it binds the first epitope with a k(off) that is at least two orders of magnitude less than the antibody's k(off) for the second epitope.

In certain methods useful herein, the competitive binding of two or more antibodies is assessed, generally by assessing the ability of one antibody to inhibit the binding of one or more other antibodies to an epitope. In certain embodiments, an antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. Generally, an antibody may be said to competitively inhibit binding of the reference antibody to a given epitope by at least 90%, at least 80%, at least 70%, at least 60%, or at least 50%.

Antibodies, or antigen-binding fragments, variants or derivatives thereof as described herein may also be described or specified in terms of their cross-reactivity. As used herein, the term “cross-reactivity” refers to the ability of an antibody, specific for one antigen, to recognize and bind to a second antigen. Generally, an observation of antibody cross-reactivity is taken as a measure of relatedness between the two (or more) different antigenic substances which support such cross-reactivity. Thus, an antibody is cross-reactive if it binds to an epitope other than the one that induced its formation.

It will be appreciated that in certain contexts, epitopes may also be referred to or described as being cross-reactive, whereby two or more different epitopes may be recognized by and/or bound by a particular antibody. The cross-reactive epitope generally contains many of the same complementary structural features as the inducing or originating epitope. In some circumstances, the cross-reactive epitope may be bound with greater affinity than the originating epitope.

It will be appreciated that for diagnostic purposes, antibodies and antigens having low or no cross-reactivity will generally be preferred.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms.

The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. The term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are fragments, derivatives, analogues, or variants of the foregoing polypeptides, and any combination thereof. The terms “fragment,” “variant,” “derivative” and “analogue” when referring to antibodies or antibody polypeptides of the present invention include any polypeptides which retain at least some of the antigen-binding properties of the corresponding native binding molecule, antibody, or polypeptide. Fragments of polypeptides of the present invention include proteolytic fragments, as well as deletion fragments. Variants of polypeptides include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Variant polypeptides may also be referred to herein as “polypeptide analogues.” Derivatives of polypeptides are considered herein as polypeptides which have been altered so as to exhibit additional features not found on the native polypeptide. Examples include fusion proteins, or functionally modified polypeptides such as PEGylated polypeptides, bead-linked polypeptides, and polypeptides covalently linked to one or more other agents or compounds. In one embodiment, a “derivative” of a polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

In one embodiment, analogues of specifically identified polypeptides will have about 70% sequence identity with sequence of the specified polypeptide, such as an amino acid sequence illustrated in the figures or fragments thereof, for example, over a sequence of 10 or more contiguous amino acids. That is, 70% of the residues of each polypeptide are the same. In a further embodiment, analogues of specifically identified polypeptides will have greater than 75% identity. In a further embodiment, analogues of specifically identified polypeptides will have greater than 80% identity. In a further embodiment, analogues of specifically identified polypeptides will have greater than 85% identity. In a further embodiment, analogues of specifically identified polypeptides will have greater than 90% identity. In a further embodiment, analogues of specifically identified polypeptides will have greater than 95% identity. In a further embodiment, analogues of specifically identified polypeptides will have greater than 99% identity. In a further embodiment, analogues of polypeptides of the invention will have fewer than about 20 amino acid residue substitutions, modifications or deletions, for example less than 10, with reference to the sequence of the specified polypeptide.

In a further embodiment, polypeptides will have greater than 70% homology. In a further embodiment, polypeptides will have greater than 75% homology. In a further embodiment, polypeptides will have greater than 80% homology. In a further embodiment, polypeptides will have greater than 85% homology. In a further embodiment, polypeptides will have greater than 90% homology. In a further embodiment, polypeptides will have greater than 95% homology. In a further embodiment, polypeptides will have greater than 99% homology. In a further embodiment, derivatives and analogues of polypeptides of the invention will have less than about 20 amino acid residue substitutions, modifications or deletions and more preferably less than 10. Preferred substitutions are those known in the art as conserved i.e. the substituted residues share physical or chemical properties such as hydrophobicity, size, charge or functional groups.

Also contemplated are analogues of polypeptides having a specified degree of identity over a specified number of contiguous amino acid residues. For example, an analogue of a specified polypeptide in one embodiment has greater than 90% amino acid sequence identity over 10 or more contiguous amino acids of the reference/specified sequence. In another embodiment, an analogue of a specified polypeptide has greater than 90% amino acid sequence identity over 20 or more contiguous amino acids of the reference/specified sequence

The term “polynucleotide” is intended to encompass a singular nucleic acid as well as plural nucleic acids, and refers to an isolated nucleic acid molecule or construct, including, for example, messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an antibody or an antigen contained in a vector is considered isolated for the purposes of the present disclosure. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides described herein. Isolated polynucleotides or nucleic acids described herein further include such molecules produced synthetically. In addition, polynucleotide or a nucleic acid may be or may include a regulatory element such as a promoter, ribosome binding site, or a transcription terminator.

As used herein, the term “sample” refers to any biological material obtained from a subject or patient, though it will be appreciated that in the majority of embodiments (for example of methods) described herein there will be a preference for samples in which the presence of one or more antibodies are capable of being present. In one embodiment, a sample can comprise blood, plasma, serum, cerebrospinal fluid (“CSF”), or urine. For example, a sample can comprise whole blood, plasma, B cells enriched from blood samples, or cultured cells (e.g., B cells from a subject). A sample can also include a biopsy or tissue sample including mucosal or neural tissue. In still other embodiments, a sample can comprise whole cells and/or a lysate of the cells. Methods for the collection and/or preparation of samples are well known in the art.

By “subject” or “individual” or “animal” or “patient” or “mammal,” is meant any subject, particularly a mammalian subject, e.g., a human patient, for whom diagnosis, prognosis, prevention, or therapy is desired.

As used herein, the terms “treat” or “treatment” refer to both therapeutic treatment and prophylactic or preventative measures, wherein the object is to prevent or slow down (lessen) an undesired physiological change or disorder, such as the development of or progression of rheumatic fever or APSGN. Beneficial or desired clinical results include, but are not limited to, alleviation of symptoms, diminishment of extent of disease, stabilized (i.e., not worsening) state of disease, delay or slowing of disease progression, amelioration or palliation of the disease state, pathogen clearance, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the manifestation of the condition or disorder is to be prevented.

In certain embodiments, the antigens described herein are used to quantitatively or qualitatively detect Streptococcus pyogenes-specific antibodies in a sample. This can be accomplished by techniques giving a visually detectable signal, which may be any one of fluorescence (immunofluorescence), a chromogenic product of an enzymatic reaction, production of a precipitate, chemiluminescence or bioluminescence. Certain embodiments employ a fluorescently or colour-labeled antibody together with light microscopy, flow cytometry, or fluorometric detection. Other techniques and labels which may be used for detecting the antibody include, but are not limited to colloidal gold, radioactive tag, GFP (green fluorescence protein), and the like, avidin/streptavidin-biotin, magnetic beads, as well as physical systems, e.g. nanotechnological system, sensitive to the actual binding.

The antigens, antibodies, or fragments thereof, may be employed in histology staining, as in immunohistochemistry, immunofluorescence or immunoelectron microscopy, as well as for in situ detection of the antibodies or proteins. Those of ordinary skill will readily perceive that any of a wide variety of histological methods, such as staining procedures can be modified in order to achieve such in situ detection.

One of the ways in which an antibody or antigen described herein can be labeled and directly detected is by linking the same to an enzyme and used in an enzyme immunoassay (EIA). This enzyme, in turn, when later exposed to an appropriate substrate, will react with the substrate in such a manner as to produce a chemical moiety which can be detected, for example, by spectrophotometric, fluorometric or by visual means. Enzymes which can be used to detectably label the antibody or antigen include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholine-esterase. The detection can be accomplished by colourimetric methods which employ a chromogenic substrate for the enzyme. In certain embodiments, detection is accomplished by visual comparison of the extent of enzymatic reaction of a substrate with similarly prepared standards, and this procedure is suitable for both soluble colour products and non-soluble colour products, including for example those implemented on nitrocellulose or plastic supports, dip strips and the like).

In some embodiments, detecting the reaction of the antibody with the antigen can be further aided, in appropriate instances, by the use of a secondary antibody or other binding partner which binds the antigen:antibody complex (being reactive with the antigen or more usually the antibody). Generally, this secondary antibody or ligand is detectably labelled, where detection of the secondary antibody allows inference of the presence of the antigen:antibody complex.

Specific binding partners, such as secondary antibodies, will frequently be reactive to a conserved region of an immunoglobulin of the species from which the sample is derived. In particularly contemplated examples herein, the secondary antibody or specific binding partner has affinity for human immunoglobulins, such as IgG. The choice of secondary antibody will be at least in part dependent on the source of the sample, and thus the nature of the antibodies expected to be present therein.

A number of well-known detection methods are suitable for use in the practice of the methods described herein. For example, enzyme immunoassays such as immunofluorescence assays (IFA), photometric assays, enzyme linked immunoabsorbent assays (ELISA), ELISPOT assay, and immunoblotting can be readily adapted to accomplish the detection of the specific antibodies.

Other detection systems which may also be used include those based on the use of protein A derived from Staphylococcus aureus Cowan strain I, protein G from group C Streptococcus (e.g., strain 26RP66), or systems which employ the use of the biotin-avidin binding reaction.

Other methods of immunoenzymatic detection in which the antigens and antibodies described herein can be employed are the Western blot, and the dot blot, in which the reagent is separated by electrophoresis and transferred to a nitrocellulose membrane or other suitable support. The sample to be tested (e.g. plasma) is then brought into contact with the membrane and the presence of the immune complexes formed is detected by the method already described. In a variation on this method, purified antigen is applied in lines or spots on a membrane and allowed to bind with any antibodies present in the sample, and the immune complexes formed are detected using the techniques described herein.

The presence of antibody:antigen complexes may also be detected by agglutination. In one representative example of such a method, the antigens described herein are used to coat, for example, latex particles to form a uniform suspension. When mixed with a sample, e.g. serum containing specific antibodies capable of recognizing the antigens, the latex particles are caused to agglutinate and the presence of large aggregates can be detected visually.

Detecting the reaction of the antibody with the antigen can be facilitated by the use of an antibody or ligand that is labeled with a detectable moiety by methods known in the art. Such a detectable moiety allows visual detection of a precipitate or a color change, visual detection by microscopy, or automated detection by spectrometry or radiometric measurement or the like. Examples of detectable moieties include fluorescein and rhodamine (for fluorescence microscopy), horseradish peroxidase and alkaline phosphatase (for either light microscopy or electron microscopy and biochemical detection and for biochemical detection by color change), and biotin-streptavidin (for light or electron microscopy). The detection methods and moieties used can be selected, for example, from the list above or other suitable examples by the standard criteria applied to such selections as is well known in the art.

While methods reliant on radioisotopes are generally viewed less favourably now than previously, radioactive detection methods which accomplish detection by radioactive labeling the antigens, antibodies or antibody fragments, and the use of a radioimmunoassay (RIA), are available. The radioactive isotope can be detected by such means as the use of a gamma/beta counter or a scintillation counter or by autoradiography.

More frequently employed methods label an antibody or antigen to be detected with non-radioactive, detectable labels, such as a fluorescent compound. When the fluorescently labeled antibody or antigen is exposed to light of the proper wavelength, its presence can be then detected due to fluorescence. Among the most commonly used fluorescent labeling compounds are fluorescein isothiocyanate, rhodamine, phycoerythrine, phycocyanin, allophycocyanin, o-phthaldehyde and fluorescamine.

The antibody or antigen can also be detectably labeled using fluorescence emitting metals such as ¹⁵²E, or others of the lanthanide series. These metals can be attached to the antibody or antigen using such metal chelating groups as diethylenetriamine pentaacetic acid (ETPA).

The antibody or antigen can also be detectably labeled by coupling it to a chemiluminescent compound. The presence of the chemiluminescent-tagged antibody or antigen is then determined by detecting the presence of luminescence that arises during a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody or antigen. Bioluminescence occurs in biological systems generally through the activity of a catalytic protein which increases the efficiency of a chemiluminescent reaction. The presence of a given bioluminescent protein is determined by detecting the presence of luminescence. Important bioluminescent compounds for purposes of labeling are luciferin, luciferase and aequorin.

Particular examples of methods for the detection of antibodies in biological samples, including methods employing dip strips or other immobilized assay devices, are disclosed for instance in the following patents: U.S. Pat. No. 5,965,356 (Herpes simplex virus type specific seroassay); U.S. Pat. No. 6,114,179 (Method and test kit for detection of antigens and/or antibodies); U.S. Pat. No. 6,077,681 (Diagnosis of motor neuropathy by detection of antibodies); U.S. Pat. No. 6,057,097 (Marker for pathologies comprising an auto-immune reaction and/or for inflammatory diseases); and U.S. Pat. No. 5,552,285 (Immunoassay methods, compositions and kits for antibodies to oxidized DNA bases).

By way of example, a microsphere assay (also called flow bead assays) also can be used to detect one or more antibodies specific for one or more Streptococcus pyogenes antigens in biological fluids (such as a blood or serum sample from a subject). This technology, as represented by systems developed by Luminex Corporation and other systems developed by Becton Dickinson, allows one to process a very small amount of sample, typically 20 μL, to detect one or several analytes. The principle of this assay is based on the coupling of a capture antibody to microspheres containing specific amounts of a red dye and an infrared dye. After incubation of these microspheres with the sample, a secondary detection antibody coupled with phycoerthrin (PE), the beads are analyzed with a flow cytometer. One laser detects the beads and a second one detects the intensity of the PE bound to those beads. This technology has been used to detect many biologically important molecules or agents, including cytokines in multiplex assays, serotyping of Streptococcus pneumoniae, simultaneous measurement of human chorionic gonadotropin (hCG) and alpha-fetoprotein (AFP), simultaneous detection of serum IgG to Toxoplasma gondii, Rubella virus, Cytomegalovirus, and Herpes Simplex Virus Types 1 and 2 (see technical notes available from Luminex Corp., for instance at their Web-site or through their catalogue)

In certain embodiments, one or more antibodies specific for one or more Streptococcus pyogenes antigens that are present in the biological sample bind to antigenic protein coupled to the microspheres. In some embodiments, the secondary detection antibody (an example of what is also referred to herein as a specific binding partner) is a monoclonal antibody, for example to human IgG. In other embodiments, the secondary detection antibody is a polyclonal antibody, for example to human IgG. Secondary antibodies used in such methods can be coupled to, for instance, biotin, or otherwise detectably labelled.

Immunological techniques suitable for uses as contemplated herein include immunological methods reliant on the formation of antibody:antigen complexes, for example using labelled antibodies or antibody fragments including labelled secondary antibodies. Exemplary methods include ELISA methods, for example indirect ELISA, competitive ELISA, sandwich ELISA, in addition to other immunologically-based methods, agents, or apparatuses, such as immunochromatographic strips, fluorescent immunomicroparticles, Western blot, biosensors based on electrochemical reactions catalyzed by enzymes attached to the antibodies, by magnetic particles coated with antibodies, by surface plasmon resonance, and other techniques in which an analyte bound to an antibody is detected. Various methods and technologies for implementing these methods exist and will be well known to a person skilled in the art, and may include microfluidic technologies, automated, and/or high throughput technologies.

Suitable assays may be quantitative techniques as ELISAs, or qualitative as rapid immunochromatographic assays, immunoblots, dip strips, or the like. It should be appreciated that assays which are usually employed as quantitative assays may in certain circumstances be employed qualitatively.

Qualitative, optionally rapid assays such as immunochromatographic assays, particularly those employing immunochromatographic strips, are particularly suited to use in areas where more complex assays technologies are unavailable or impractical, for example in developing countries, or as a first line diagnosis to identify subjects who may benefit from further assessment.

For example, in one embodiment of a qualitative assay in which it is seen if the presence or amount of one or more antibodies specific for one or more Streptococcus pyogenes antigens is greater or less than a certain amount, a qualitative ELISAs assay using two or more antigens as described herein is used. The procedure is carried out with a kit as described herein, such as a kit that contains a composition, such as a buffer solution or a sample preparation solutions which includes one or more Streptococcus pyogenes antigens, optionally a negative control in which no antibodies specific for a Streptococcus pyogenes antigen is present, optionally a positive control in which one or more antibodies specific for one or more Streptococcus pyogenes antigens are present, and the components of an ELISA such as a reaction vessel, for example a multi-well plate, and one or more secondary detection antibodies to detect the formation of a complex between the one or more antibodies specific for one or more Streptococcus pyogenes antigens and the one or more Streptococcus pyogenes antigens, and optionally one or more components to immobilize the complex and/or for the development of the assay. In one embodiment, the composition comprising the one or more Streptococcus pyogenes antigens is first introduced to the reaction vessel, such as the ELISA plate under conditions to immobilize the one or more antigens onto the vessel. In another embodiment, the reaction vessel such as the ELISA plate is provided with one or more Streptococcus pyogenes antigens already immobilized thereon. In a further specifically contemplated example, the one or more secondary detection antibodies are either already immobilized on or in the reaction vessel, or are provided in a composition and introduced into the reaction vessel under conditions to immobilize the detection antibodies thereto. It will be appreciated by those skilled in the art that various alternative methods exist whereby analyte-dependent complex formation, capture and labelling provides a detectable signal, and that these methods are generally dependent on the immobilization of an antibody:antigen complex to allow detection.

In another embodiment of a qualitative assay in which the presence of one or more antibodies specific for one or more Streptococcus pyogenes antigens is determined, or in which it is determined whether the amount of said antibodies is greater or less than a certain threshold, an immunochromatographic strip is used. In one example, the strip comprises two or more antigens as described herein, and will generally be provided with one or more compositions, such as a buffer solution or a sample preparation solutions, optionally a negative control in which no antibodies to a Streptococcus pyogenes antigen is present, optionally a positive control in which one or more antibodies specific for one or more antigens are present, and a specific binding partner to detect the formation of a complex between the one or more antibodies specific for one or more Streptococcus pyogenes antigens and the one or more Streptococcus pyogenes antigens, and optionally one or more components for the development of the assay. In one example, a separate immunochromatographic strip is provided, in which no Streptococcus pyogenes antigens are present, to act as a negative control. In other embodiments, one or more immunochromatographic strips has at least two different regions, one of which comprises one or more Streptococcus pyogenes antigens, optionally another of which comprises no Streptococcus pyogenes antigens, and optionally another of which comprises one or more immobilized antibodies or other agents capable of being bound by the secondary detection antibody or specific binding partner, so as to act as a positive control region.

In one particular embodiment, detection is effected through capture ELISA. Capture ELISA (also known as “sandwich” ELISA) is a sensitive assay to quantify very small (picogram to microgram) quantities of substances (such as hormones, cell signaling chemicals, infectious disease antigens and cytokines, and in the context of this disclosure, antibodies). This type of ELISA is commonly considered when the substance to be analyzed may be too dilute to bind to a support material, such as a polystyrene microtiter plate (such as a protein in a cell culture supernatant) or does not bind well to plastics (such as a small organic molecule). Optimal dilutions for the capture reagent (for example, a capture antigen), samples, controls, and detecting antibodies as well as incubation times are generally determined empirically and may require extensive titration. Ideally, one would use an enzyme-labeled detection antibody or detection antigen. However, if the detection antibody or antigen is unlabeled, the secondary antibody should not cross-react with the coating antibody or antigen.

As used herein in the specification, the terms “detectable moiety”, “detectable label’, and grammatical equivalents, refers to any atom, molecule or a portion thereof, reagent, or agent, the presence, absence or level of which may be monitored directly or indirectly. One example includes radioactive isotopes. Other examples include (i) enzymes which can catalyze color or light emitting (luminescence) reactions and (ii) fluorophores. The detection of the detectable moiety can be direct provided that the detectable moiety is itself detectable, such as, for example, in the case of fluorophores. Alternatively, the detection of the detectable moiety can be indirect. In the latter case, a second moiety which reacts with the detectable moiety, itself being directly detectable is typically employed. The detectable moiety may be inherent to the antibody or labelled antigen, for example by covalent linkage. For example, the constant region of an antibody can serve as an indirect detectable moiety to which a secondary antibody having a direct detectable moiety can specifically bind.

Thus, secondary antibodies are particularly suitable means for the detection of the antibody in the methods described herein. This secondary antibody may be itself conjugated to a detectable moiety. One of the ways in which an antibody in accordance with the present invention can be detectably labeled is by linking the same to an enzyme. This enzyme, in turn, when later exposed to an appropriate substrate, will react with the substrate in such a manner as to produce a chemical moiety which can be detected, for example, by spectrophotometric, fluorometric or by visual means. Enzymes which can be used to detectably label the antibody include, but are not limited to, horseradish peroxidase, alkaline phosphatase, malate dehydrogenase, staphylococcal nuclease, delta-5-steroid isomerase, yeast alcohol dehydrogenase, alpha-glycerophosphate dehydrogenase, triose phosphate isomerase, asparaginase, glucose oxidase, beta-galactosidase, ribonuclease, urease, catalase, glucose-6-phosphate dehydrogenase, glucoamylase and acetylcholinesterase.

The detection can be accomplished by colourimetric methods, which employ a chromogenic substrate for the enzyme. Detection may also be accomplished by visual comparison of the extent of enzymatic reaction of a substrate in comparison with similarly prepared standards.

In embodiments (for example, of methods, assays, and kits) which employ a solid support to which a reagent is bound—for example, an antigen or antibody as applicable, the solid support is any water-insoluble, water-insuspensible, solid support. Examples of suitable solid support include beads, e.g., of polystyrene, filter paper, test tubes, dipstrips, and microtiter plates. The bound reagent may be bound to the solid support by covalent bonds or by adsorption. The advantage of the use of a solid support is that no centrifugation step is needed for the separation of solid and liquid phase, although centrifugation can be employed in particular embodiments, for example, bead-based assays, to facilitate processing.

The solid support mentioned above can include polymers, such as polystyrene, agarose, Sepharose, cellulose, glass beads and magnetizable particles of cellulose or other polymers. The solid-support can be in the form of large or small beads or particles, tubes, plates, strips, or other forms.

As a solid support, use is commonly made of a test tube or a microtiter plate, the inner walls of which are coated with a first antibody or antigen, for example, the antigens specific to, or of any fragment or derivative thereof, the Streptococcus pyogenes antibodies to be detected, such as those antigens specifically disclosed herein.

The provision of a kit comprising one or more components necessary to carry out one or more of the methods described herein is also contemplated. The kit will typically comprise a composition comprising at least one of the Streptococcus pyogenes antigens described herein, for example a composition comprising two or more of said antigens. Other components for carrying out a method in accordance with this disclosure will usefully be included in the kit. For example, the kit may comprise one or more reagents for constituting the medium favourable for contacting the one or more antigens with a biological sample. The kit may also include equipment for sample collection, such as a swab, a pipette, or similar collection means, and equipment for carrying out one or more reaction steps where the reagents are brought into contact, such as an incubation means including a liquid or semisolid medium placed in a plate, test tube, a glass or plastic surface, a well, or on a strip of absorbent paper, or similar means. Other kit components can include one or more reagents enabling the detection of a complex formed between the one or more antigens and one or more Streptococcus pyogenes antigen specific antibodies present in a biological sample.

The methods, assays and kits contemplated herein may in certain embodiments include or utilise the contacting of one or more samples with the other assay reagents (such as the one or more Streptococcus pyogenes antigens) with the latter arranged in an array, for example to allow rapid handling of multiple samples, including high-throughput implementations.

The term “array” as used herein refers to an “addressed” spatial arrangement of the recognition-agent(s), such as the combination of two or more Streptococcus pyogenes antigens. Each “address” of the array is a predetermined specific spatial region containing one or more recognition agents. For example, an array may be a plurality of vessels (test tubes), plates, micro-wells in a micro-plate, each containing a set of antigens. Various implementations of arrays are envisaged. In one embodiment, each address comprises similar or identical recognition agents, and multiple samples are assayed, one at each address. In another embodiment, each address comprises a different recognition agent or different combination of recognition agents, and aliquots of a single sample are introduced to each address. In other embodiments, a combination of these approaches is taken. An array may also be any solid support holding in distinct regions (dots, lines, columns) known recognition agents, for example where different recognition agents are disposed in different combinations at one or more different locations, or where different samples are contacted at different locations. In certain embodiments, the array includes built-in appropriate controls, for example, regions without the sample, regions without the antigen, regions without either, (e.g., with solvent and reagents alone), and regions containing synthetic or isolated antibodies bound to the one or more antigens as a positive control.

Solid supports useful herein, for example for an array or a kit is typically substantially insoluble in liquid phases. Solid supports of the current invention are not limited to a specific type of support. Rather, a large number of supports are available and are known to one of ordinary skill in the art. Thus, useful solid supports include solid and semi-solid matrixes, such as aerogels and hydrogels, resins, beads, biochips (including thin film coated biochips), microfluidic chip, a silicon chip, multi-well plates (also referred to as microtitre plates or microplates), membranes, filters, conducting and nonconducting metals, glass (including microscope slides) and magnetic supports. More specific examples of useful solid supports include silica gels, polymeric membranes, particles, derivatized plastic films, glass beads, cotton, plastic beads, alumina gels, polysaccharides such as Sepharose, nylon, latex beads, magnetic beads, paramagnetic beads, superparamagnetic beads, starch and the like.

### Antigens

It will be appreciated that in principle any Streptococcus pyogenes antigen to which antibodies are generated in vivo in a subject can be used in the methods described herein, and in particular the multiplex methods described herein. As shown herein in the Examples, clinically established antigens DNaseB and SLO can be crosslinked to beads for use in multiplex CBA assays. In certain cases, and without wishing to be bound by any theory, the applicants have established that certain antigens, for example SpnA, can usefully be stabilized to facilitate their inclusion in the multiplex assays described herein. In the case of SpnA, truncation of the full-length polypeptide improved immunological stability, particularly when crosslinked or otherwise bound to the solid support (in this case beads) used in implementations of the methods described herein.

In one embodiment, two or more, and in particularly contemplated examples three or more Streptococcus pyogenes antigens are utilised, for example, in a single, multiplex assay. In one example, each antigen is bound to beads or another substrate wherein each population of antigen-bound beads is distinguishable from the other. One example of such a multiplex assay is presented herein in the Examples, where SLO, DNaseB, and SpnA are each covalently linked to different populations of beads such that each population of antigen-labelled bead is separately identifiable, and as a consequence, the amount of antibody specific to each antigen can be determined in a single assay.

In certain embodiments, the two or more Streptococcus pyogenes antigens are provided in a single composition—for example, a single solution comprising any required buffers or carriers, stabilizers, or the like—for example, when brought into contact with sample, and are thus particularly suited for multiplex implementation. In one example, Streptococcus pyogenes SpnA antigen together with at least one other Streptococcus pyogenes antigen is provided, for example, SpnA and at least one other Streptococcus pyogenes antigen is present in a single composition, such as when brought into contact with sample. In a specifically contemplated example, Streptococcus pyogenes SpnA antigen and SLO are provided, for example, in a single composition, such as when brought into contact with sample. In another specifically contemplated example, Streptococcus pyogenes SpnA antigen and DNaseB are provided, for example, in a single composition, such as when brought into contact with sample. In a further specifically contemplated example, Streptococcus pyogenes SpnA antigen, DNaseB, and SLO are provided, for example, in a single composition, such as when brought into contact with sample.

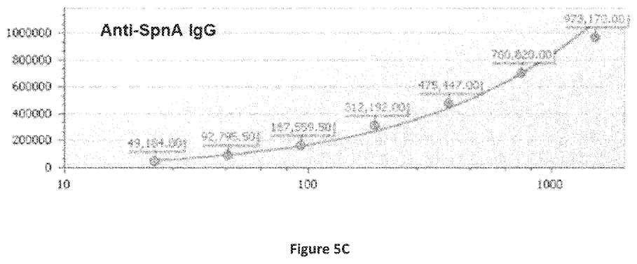

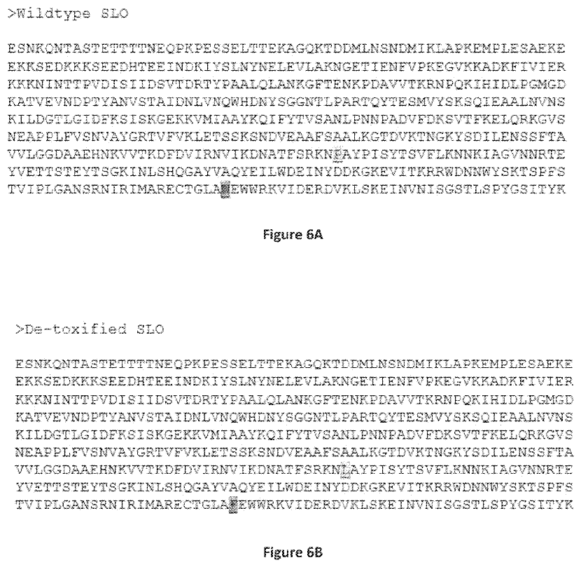

SLO (AAK33267.1) is a secreted, pore forming cytolysin. In one embodiment, SLO is derived from Streptococcus pyogenes M1 GAS (strain: SF370, serotype: M1, gene locus tag: SPy_0167 (NCBI: NP_268546.1)). In one example, a recombinant fragment of SLO is used, for example a recombinant polypeptide comprising amino acids 34-571 (see FIG. 6A, SEQ ID NO. 1). In one example, the recombinant polypeptide is tagged, for example to aid purification, and N-terminal or C-terminal His tags are particularly contemplated. In another embodiment, a ‘detoxified’ SLO analogue is used, in which one or more amino acids determined to contribute to toxicity are substituted for amino acids resulting in a polypeptide that is immunologically cross-reactive with wild-type SLO, but exhibits reduced toxicity (for example to reduce production, handling or containment constraints or requirements). The amino acid sequence of a representative example of a detoxified SLO analogue is presented in FIG. 6B and SEQ ID NO. 2. In this example of a detoxified SLO, two point mutations in the encoding nucleotide sequence result in P427L and W535F substitutions: the substituted amino acids are underlined in FIGS. 6A and 6B.

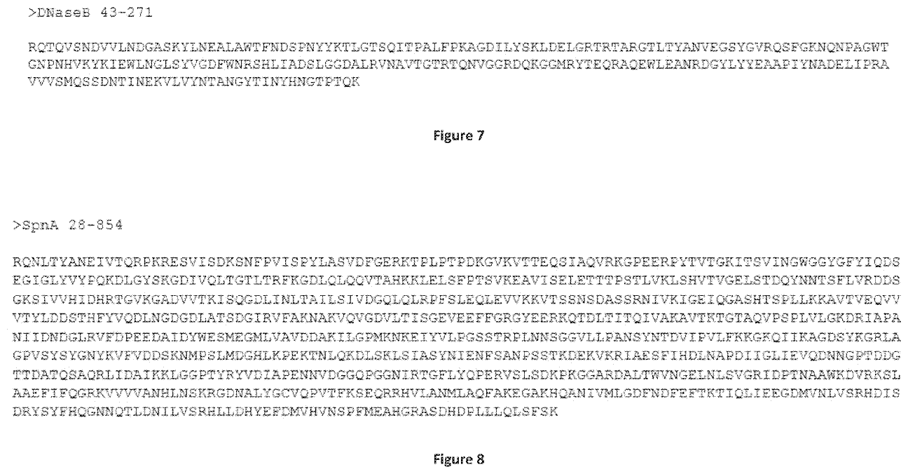

DNase-B (AAK34710.1) is a secreted enzyme that degrades DNA. In one embodiment, DNaseB is derived from Streptococcus pyogenes M1 GAS (strain: SF370, serotype: M1, gene locus tag: SPy_2043 (NCBI: NP_269989.1)). In one example, a recombinant fragment of DNaseB is used, for example a recombinant polypeptide comprising amino acids 43-271 (see FIG. 7, SEQ ID NO. 5). In one example, the recombinant polypeptide is tagged, for example to aid purification, and N-terminal or C-terminal His tags are particularly contemplated.

SpnA (AAK33693.1) is a cell wall-anchored enzyme that also degrades DNA. In one embodiment, SpnA is derived from Streptococcus pyogenes M1 GAS (strain: SF370, serotype: M1, gene locus tag: SPy_0747 (NCBI: NP_268972.1)). In one example, a recombinant fragment of SpnA is used, for example a recombinant polypeptide comprising amino acids 28-854 (see FIG. 8, SEQ ID NO. 8). In one example, the recombinant polypeptide is tagged, for example to aid purification, and N-terminal or C-terminal His tags are particularly contemplated.

Those skilled in the art will recognise that in certain embodiments the invention disclosed herein, including the methods, polypeptides, beads, compositions and kits as herein described and particularly embodiments relating to multiplex diagnostic methods and compositions described herein, provides for the accurate and rapid diagnosis and/or identification of patients who require treatment for recent-onset rheumatic fever or acute PSGN, or for acute Streptococcus pyogenes infection, but who will not require long-term treatment, including long-term prophylactic treatment, for rheumatic fever, PSGN, or persisting Streptococcus pyogenes infection, or who will require only ongoing monitoring, for example to rapidly identify subsequent Streptococcus pyogenes infection.

In various embodiments, for example when using a method as herein described, a patient is identified as having or having had a recent exposure to Streptococcus pyogenes or a recent Streptococcus pyogenes infection, that patient will undergo both treatment suitable for the treatment of recent Streptococcus pyogenes infection, and ongoing treatment, such as prophylactic treatment to prevent subsequent Streptococcus pyogenes infection, or to ameliorate one or more symptoms of rheumatic fever, PSGN, chronic rheumatic heart disease, or to monitor disease status. In other embodiments, when using a method as herein described a patient is identified as having or having had a prior exposure to Streptococcus pyogenes or a prior Streptococcus pyogenes infection, that patient will undergo ongoing treatment, such as prophylactic treatment to prevent subsequent Streptococcus pyogenes infection, or to ameliorate one or more symptoms of rheumatic fever, PSGN, chronic rheumatic heart disease, or to monitor disease status. Representative treatments include those described herein in relation to

The following examples, sequence listing and figures are provided to aid the understanding of the present invention, the true scope of which is set forth in the appended claims. It is understood that modifications can be made in the procedures set forth without departing from the spirit of the invention.

### EXAMPLES

### Example One

This example describes the development of a bead-based multiplex assay to determine the presence and amount of GAS-specific antibodies in biological samples.

### Methods

### Study Subjects

In this study, human serum was obtained from four different sources. Patients with ARF were diagnosed according to the New Zealand modification of the Jones criteria [3] and recruited while hospitalized at Starship Children's Hospital in Auckland between 2004-2006 (n=8) and Waikato Hospital in Hamilton between 2012-2015 (n=8). Sera from ethnically matched healthy children, aged 6, were obtained from the Children of Scope (CoS) study. Finally, sera were obtained from unmatched, healthy volunteers aged 20 years or older recruited at the University of Auckland as an additional control group. Demographics are shown in Table 1. All participants had provided written informed consent and appropriate ethical board approval was obtained for each of the four sites.

TABLE 1
Study participants*

| | ARF (n = 16) | Healthy Children (n = 13) |
|---|---|---|
| Sex(n) | | |
| Male/Female | 11/5 | 7/6 |
| Age (years) | | |
| Mean | 10.3 | 6 |
| Range | 6-15 | 6-6 |
| Ethnicity | | |
| Maori/Pacific | 13/3 | 11/2 |

*The healthy adult group (n = 18) were comprised of adult volunteers >20 years of age with no recent history of pharyngitis. Ethnicity data were not available for these participants.

### Antigen Preparation

The recombinant antigens utilized in this study were all prepared in their mature form without N terminal signal sequences. SLO, with a molecular weight of 64.4 kDa (amino acids 34-571, SEQ ID NO. 1) and a N-terminal Hiss-tag was purchased from Fitzgerald Industries International. The gene encoding DNase B was amplified from S. pyogenes SF370 (ATCC 700294) genomic DNA using the following primers: forward, 5′ CACCATGCGACAAACACAGGTCTCAAATGATGTTG-3′ [SEQ ID NO. 3] and reverse, 5′ TTTCTGAGTAGGTGTACCGTTATGGTAGTTAATGG-3′ [SEQ ID NO. 4] for cloning into pET101/D TOPO (Life Technologies) using Topo cloning methodology. The resulting vector encodes DNaseB (amino acids 43-271, amino acid sequence depicted herein as [SEQ ID NO. 5]) followed by a C-terminal Hiss-tag for a total molecular weight of 29 kDa. The protein was expressed in Escherichia coli BL21 λDE3 cells with 1 mM IPTG induction at 37° C. for 3 h in Lennox broth (LB) media supplemented with ampicillin and 0.1% glucose. SpnA was amplified from S. pyogenes SF370 genomic DNA with primers that contained KasI and XhoI restriction enzyme sites (underlined): forward, 5′ AAAGGCGCCCGCCAAAATTTGACTTATGCCAA-3′ [SEQ ID NO. 6] and reverse, 5′ AAACTCGAGCTATTTGGAAAATGATAATTGAAGTAACA-3′ [SEQ ID NO. 7]. The resulting spnA amplicon (encoding SpnA amino acids 28-854, [SEQ ID NO. 8]) was cloned into a pProExHta vector that encodes an N terminal Hiss-tag and transformed into E. coli BL21 λDE3 cells. Protein expression was induced with 0.3 mM IPTG at 18° C. for 16 h in LB containing ampicillin. Both rDNaseB and rSpnA were purified from E. coli cell lysate using standard Ni²⁺-NTA affinity chromatography. The Hiss-tag was cleaved off rSpnA using recombinant tobacco etch virus protease (1:100 ratio of rTEV to recombinant protein) to yield an 85 kDa protein. The purity of all antigens was verified by SDS-PAGE.

### Purification of Antigen Specific IgG

IgGs specific for SLO, DNaseB and SpnA were purified from pooled human immunoglobulin (intravenous immunoglobulin, IVIG (Intragam® P)). Affinity columns for each of the GAS antigens were generated by covalently coupling the antigens to agarose resin via their primary amines using an AminoLink® Coupling kit (Thermo Scientific). A 5 ml solution of IVIG was diluted four-fold with phosphate buffered saline (PBS) (pH 7.4) and passed over the resin to allow antibody binding. The resin was washed four times with PBS to remove unbound antibody. Bound antibody was eluted using 0.2M glycine-HCl buffer (pH 2.5-3.0) and immediately neutralized with 1M Tris buffer (pH 9). Trace IgA was removed using Melon spin columns (Thermo Scientific) and the resulting antigen-specific IgG was concentrated using a centrifugal filter (Merck Millipore). To confirm that the eluted IgG was specific for the antigen it was isolated against, Enzyme-Linked Immunosorbent Assays (ELISA) were performed. Plates were coated with antigen at 5 μg/ml and blocked with PBS supplemented with 0.1% Tween-20 and 5% skim milk powder (PBST-5% milk) for 1 h at room temperature (RT). Purified IgG was added for 1 hour at RT and binding was detection using an anti-human horseradish peroxidase (HRP) secondary antibody (1:3000; Santa Cruz Biotechnology) as previously published [4].

### Cytometric Bead Array Assay

Each of the antigens was coupled to functional beads using an amine-to-sulfhydryl crosslinker, sulfo-SMCC, according to the manufacturer's instructions (Becton, Dickinson and Company). Briefly, color-coded 7.5 am polystyrene beads were prepared for conjugation by adding 25 mM dithiothreitol (DTT). The target antigen (90 μg) was modified by adding 44 μg/ml sulfo-SMCC solution and unreacted protein was removed using a Bio-Rad spin column (Biorad). The modified protein and functional beads were then mixed and incubated at room temperature for 1 h before adding N Ethylmaleimide (44 μg/ml) and incubating for a further 15 min. The washed, conjugated beads were stored at 4° C. protected from light. The antigens were conjugated to functional beads that contain differing ratios of fluorophores (APCy7 and APC) to ensure fluorescence at unique positions using two detectors (FL3 and FL4) on a flow cytometer. Bead positions were selected to ensure maximise separation between the antigens as follows: rSLO, position E4; rDNaseB, position A4; and rSpnA, position A9.

Incubations for Cytometric Bead Array (CBA) experiments were conducted in duplicate in 96-well U-bottom plates. Singleplex assays were performed by incubating rSLO-, rDNaseB and rSpnA-coated beads separately for 1 hour at RT with serum samples diluted 1:10,000 in assay diluent. To detect serum antibody binding R-Phycoerythrin-conjugated donkey anti-human IgG Fcγ-specific antibody (Jackson ImmunoResearch) was added at a concentration of 1:100 for 2 h at RT. The median fluorescence intensity (MFI) for each reaction was read on a flow cytometer (BD Accuri C6). Multiplex bead assays were performed by following the same protocol except the rSLO-, rDNaseB- and rSpnA-coupled beads were mixed at equal ratios prior to adding sera samples diluted 1:10,000. A 1:30 dilution of donkey anti-human IgG Fcγ-specific antibody was used in the multiplex assays for detection.

A seven-point standard curve was created for each antigen by mixing known starting concentrations of SLO-, DNaseB- and SpnA-specific antibody in one tube and performing a two-fold dilution series. A starting concentration of 500 ng/ml was used for SLO and DNaseB, whereas 1500 ng/ml was used for SpnA. The beads were incubated with each of the diluted standards for 1 h, followed by detection with donkey anti-human IgG Fcγ-specific secondary antibody as described above. MFI values were converted into concentration (g/ml) and a five-parameter logistic regression equation was used to generate a standard curve for each antigen using Flow Cytometric Analysis Program (FCAP) Array software, version 3 (BD). When study subject sera were run in a multiplex assay alongside the seven-point standard curves for each antigen, MFI was converted into concentration (g/ml) using the FCAP Array software. The lower limit of detection for each antigen was defined as the lowest concentration on the standard curve whose MFI was greater than 3 standard deviations above the blank (where the blank is beads plus secondary) as previously published [5].

### Data Analysis and Statistics

Upper limit of normal (ULN) values were calculated for each antigen by ranking the antibody concentrations determined for each of the CoS sera samples and determining the 80th centile in Microsoft Excel (version 15.24). Statistical analysis and graphs were prepared using GraphPad Prism (version 7a). All correlations were analysed using linear regression.

### ASO and ADB Titres Using Commercial Assays

Both ASO and ADB titres were determined at Labtests Pathology, Auckland, New Zealand. ASO titers (IU/mL) were measured by a turbidimetric technique using the human anti streptolysin-O kit on a SPAplus analyser (The Binding Site, CA, USA). ADB titers (U/mL) were measured by an enzyme inhibition assay (bioMerieux, Marcy l'Etoile, France). This assay provides an inexact figure for low titers of <100 U/ml; a midtiter value of 50 U/mL was estimated for samples that fell in this range.

### Results

### Bead Coupling and Multiplex Assay

To generate GAS antigen-coupled beads for analysis in this study, highly purified preparations of each of the three antigens were produced recombinantly in E. coli. The secreted proteins (SLO and DNaseB) were expressed without their N-terminal signal sequence and SpnA was expressed without the N-terminal signal sequence and truncated at K854 upstream of the sortase motif for improved protein stability. Each of the three proteins was coupled to a functional CBA bead that fluoresced in a unique position: rSLO (position E4), rDNaseB (position A4) and rSpnA (position A9). The bead positions were chosen to ensure maximum separation on a two-colour fluorescence plot. Various serum dilutions and concentrations of the anti-IgG detection reagent were tested to determine the linear range and saturation point of the assay. This trial and error process identified a 1:10,000 serum dilution as being in the linear range for all three antigens.

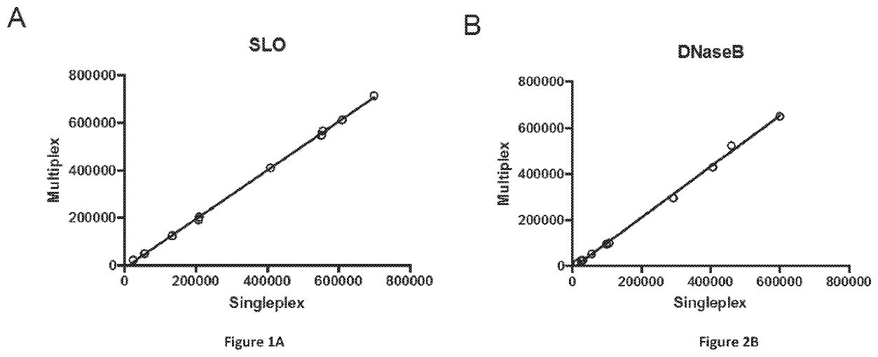

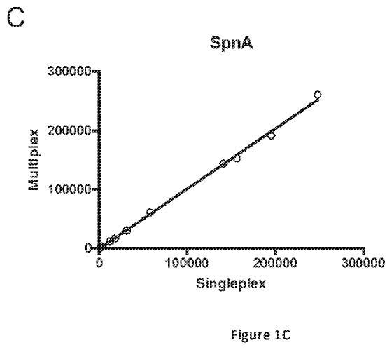

In order to assess whether the titres to the three antigens could be measured simultaneously the results of singleplex assays were compared with multiplex assays. Singleplex assays were performed in which each bead was incubated with sera from 10 participants diluted 1:10,000. These 10 sera were a mix of ARF and control samples that were chosen as previous ELISA had shown the reactivity of these participants against the three antigens ranged from low to high, ensuring a good spread of MFI in CBA assays (FIG. 1). These same 10 sera were then tested in multiplex assays in which the three antigen beads were mixed in equal parts and incubated with the test sera in a single assay well. The MFI in these multiplex assays showed an extremely strong correlation with the singleplex MFI for each antigen as shown in FIG. 1 (R² values: SLO=0.999; DNaseB B=0.998; and SpnA=0.998). This indicates there was no interference or IgG cross-reactivity between the beads and demonstrates the feasibility of a multiplex assay comprising the three streptococcal antigens.

### Standardization and Precision of the Multiplex CBA Assay

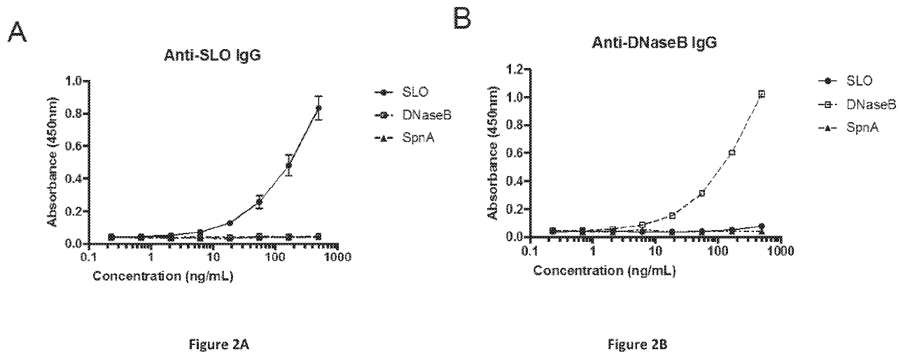

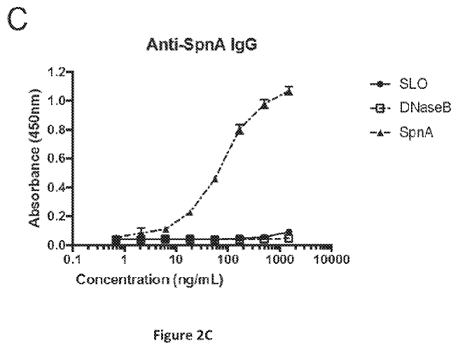

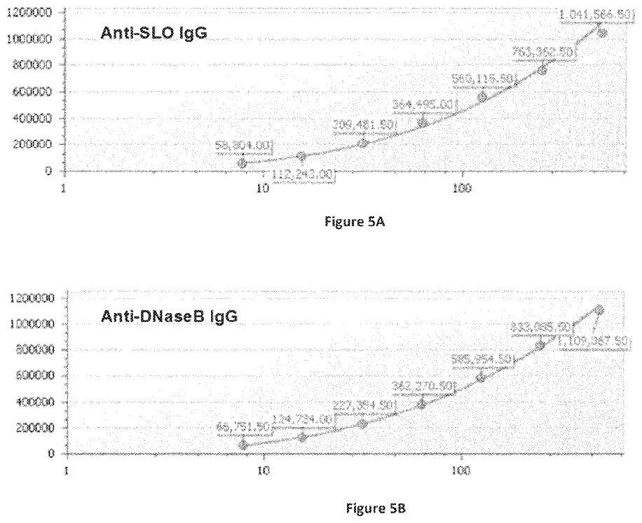

Standard curves for each of the three antigens were generated to determine the concentration of antibodies binding the antigen-coupled beads and enable comparison between assay runs. IgG specific for SLO, DNaseB and SpnA were purified from IVIG by affinity chromatography. The specificity of the purified antibodies was verified by ELISA. As shown in FIG. 2, the SLO-, DNaseB- and SpnA-specific antibodies only showed reactivity to their corresponding antigen and no detectable reactivity with the other two antigens. The purified IgG was used to generate seven-point standard curves for each antigen. Known concentrations of purified IgG were diluted twofold with starting concentrations of 500 ng/ml for anti-SLO and anti-DNase B and 1500 ng/ml for anti-SpnA. These diluted standards were incubated with the antigen-coupled beads in a multiplex format and standard curves were fitted using a five-parameter logistic formula. Example standard curves are shown in FIG. 5. The standard curves were highly reproducible with a fitting accuracy of at least 98% demonstrating the utility of affinity purified, polyclonal antibodies as reference standards for these antigens.

The purified antibody standards were used to determine lower limits of detection for the three antigens in the CBA assay: anti-SLO, 1 ng/mL; anti-DNaseB, 0.1 ng/mL; and anti-SpnA 0.1 ng/mL. The Coefficient of Variability (CV) for the multiplex assay was assessed using the same 10 sera utilised in the singleplex/multiplex comparison above. The concentrations of IgG for these 10 sera were measured in assays incorporating the IgG standard curves and the average intra- and inter-assay CVs were <4% and <15%, respectively, for each of the antigens (Table 2). These CVs demonstrate the multiplex bead-based assay has good precision and is repeatable. The reproducibility of both the standard curve and the assay test results means these reagents can be utilised to check the coupling efficacy and integrity of future batches of antigen-coupled beads.

TABLE 2
Coefficients of Variability

| | SLO | DNaseB | SpnA |
|---|---|---|---|
| Intra-assay CV (%) | 3.05 | 3.44 | 3.24 |
| Inter-assay CV (%) | 11.17 | 12.32 | 12.03 |

### Measuring Antibody Titres in ARF Patient Sera

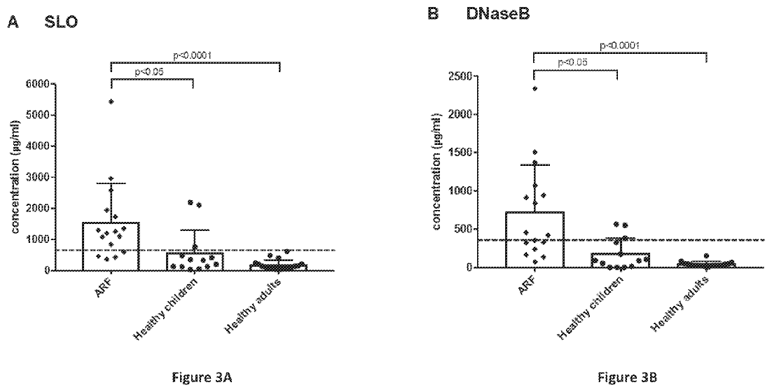

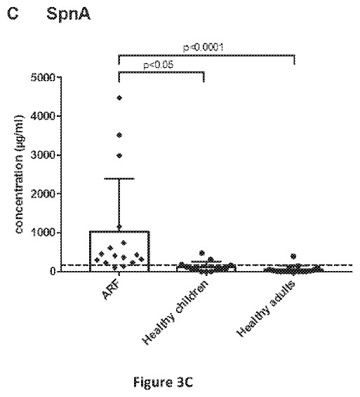

To assess the utility of the SpnA antigen and bead-based technology in clinical streptococcal serology, the multiplex assay was run on all study subjects (Table 1). The concentration of IgG specific for SLO, DNaseB and SpnA could be determined for all 47 participants in one experiment, performed by a single operator, on 1-day. As shown in FIG. 3, the mean antibody titres in ARF samples were significantly higher than the mean titres in both the healthy children and healthy adult control groups for each of the three antigens. In keeping with observations in previous studies [6], the titres for ASO and ADB were higher and showed more spread in healthy children than in healthy adults as illustrated by the larger confidence intervals in Table 3. Notably, the titres for SpnA in healthy children were similar to those in healthy adults, and had narrower confidence intervals compared to those for ASO and ADB in the healthy children group (Table 1). This supports previous observations that background titres for SpnA are low in healthy individuals [2].

The ability of the antigens to detect a previous GAS exposure for ARF diagnosis was assessed using ULN values. The ULN, or 80th centiles, were calculated from the healthy children group as 644, 360 and 170 μg/ml for SLO, DNaseB and SpnA, respectively. The lower ULN for SpnA reflects the reduced titres seen in the healthy children compared with SLO and DNaseB. These experimentally determined cut-offs, shown as a dotted line in FIG. 3, were then applied to the ARF samples to determine the sensitivity of each antigen. This is the number of true positives that were detected based on whether an observed titre was above the ULN. DNaseB was the least sensitive detecting just 9 out of 16 ARF samples (56.25%). SLO showed intermediate sensitivity by detecting 12 out of 16 ARF samples (75%). SpnA showed the highest sensitivity detecting 14 out of 16 ARF samples (87.5%).

TABLE 3
Summary statistics for the concentration of antibodies (μg/ml)
specific for SLO, DNaseB, and SpnA determined by Cytometric Bead Assay

| | Acute rheumatic fever | Healthy children | Healthy adults |
|---|---|---|---|
| Antigen | (n = 16) | (n = 13) | (n = 18) |
| Streptolysin-O | | | |
| Mean (95% CI) | 1539 (859.9-2218.0) | 559 (115.2-1003.0) | 173 (89.6-256.2) |
| Median (95% CI) | 1225 (601.4-1948.0) | 326 (123.7-757.9) | 123 (53.6-201.4) |
| DNaseB | | | |
| Mean (95% CI) | 718.5 (387.4-1050) | 180.9 (56.2-305.5) | 41.7 (24.7-58.7) |
| Median (95% CI) | 439.5 (240.9-1071) | 89.01 (0-383.7) | 29.38 (22.8-49.6) |
| SpnA | | | |
| Mean (95% CI) | 1029 (304.9-1752) | 119.8 (36.7-202.9) | 52.2 (1.3-103.1) |
| Median (95% CI) | 422.2 (229.1-1156) | 76.01 (0-177.3) | 7.4 (0-49.7) |

Comparison with Existing Serological Tests

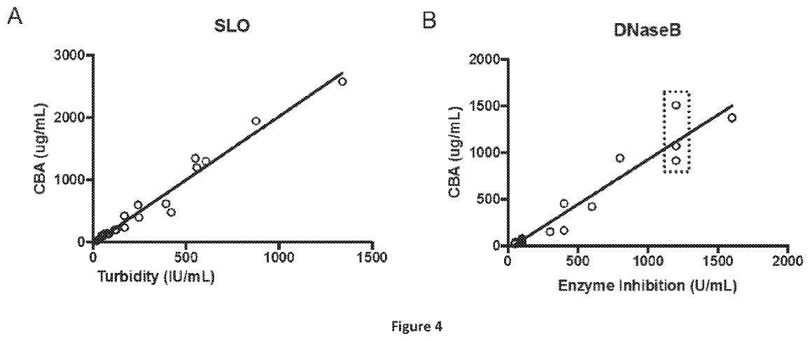

To compare the multiplex CBA assay with existing, commercially available methodology, sera from 20 participants for which sufficient volumes were available were subjected to ASO and ADB testing at a commercial laboratory. ASO was measured using the widely employed turbidimetric technique and exact values in international units (IU/mL) were obtained. In contrast the ADB titres were measured using an enzyme inhibition assay that provides titre ranges (100, 200, 300, 400, 600, 800, 1200 and 1600). As shown in FIG. 4A, there was an excellent correlation between the concentration of ASO IgG determined in the CBA assay and the commercially available turbidimetric technique (R²=0.968). As shown in FIG. 4B, there was also a good correlation between the concentration of ADB IgG determined in the CBA assay and the commercially available enzyme inhibition assay (R²=0.934).

However, the lack of precision of the ADB enzyme inhibition assay is also illustrated in the figure. Three samples were classified as ‘1200’ in the enzyme inhibition assay, yet the concentration of anti-DNaseB IgG measured in our CBA assay was 1508, 1070 and 914 μg/ml, respectively (boxed data points).

### Discussion

This example presents the preparation of a multiplex bead-based assay for GAS serology, and the use of this assay in determining antibody concentrations in a range of samples from healthy and ARF subjects. Usefully, this example demonstrates that three Streptococcus pyogenes antigens can be used in combination in a single multiplex assay, to identify the presence of distinct populations of Streptococcus pyogenes-specific antibodies, with no significant cross-reactivity, using very small sample volumes (1 μL or less).

The multiplex assay presented herein can be employed quantitatively for each of the antigen:antibody complexes, unlike existing DNaseB assays in particular. Moreover, improved sensitivity/specificity is expected, in part due to the inclusion of SpnA which has a lower background in healthy subjects, and in part due to improved sensitivity, which notably is not negatively impacted by the multiplex implementation.

### Example Two

This example describes the development of a bead-based multiplex assay using the Luminex platform to determine the presence and amount of GAS-specific antibodies in biological samples.

### Methods

### Study Subjects

In this study, human serum was obtained as described in Example One above. Again, all participants had provided written informed consent and appropriate ethical board approval was obtained.

### Serum Preparation

Sera samples were collected and diluted 1:15,000 for analysis. The IVIG reference was diluted 1:60,000 prior to use.

### Luminex Bead Preparation and Assay

Each of the antigens was coupled to xMAP microspheres using an amine-to-carboxyl crosslinker, according to the manufacturer's instructions (Luminex Corporation). Three different antigen:bead ratios were trialled for conjugation, and beads conjugated with 12.5 μg antigen were selected for further analysis.

Three well separated bead positions were selected, with DNAseB at bead position 030; SLO at bead position 072; and SpnA at bead position 078. Conjugated beads were incubated with a 1:30 dilution of anti-human IgG secondary antibody for multiplex reactions.

Luminex assays were conducted on a Magpix™ system (Merck), in accordance with the manufacturer's instructions.

### Results

### Singleplex and Multiplex Assays

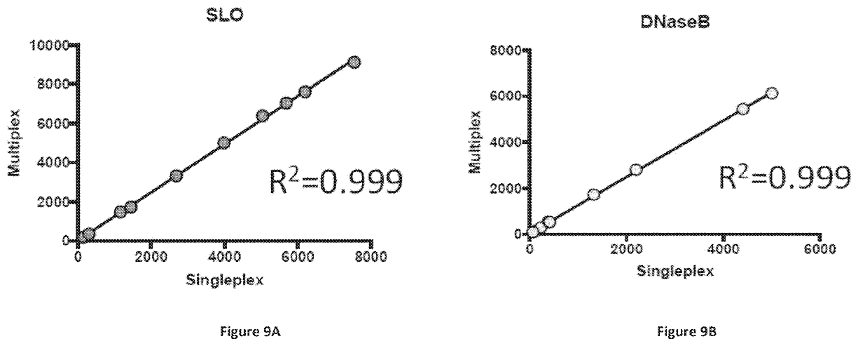

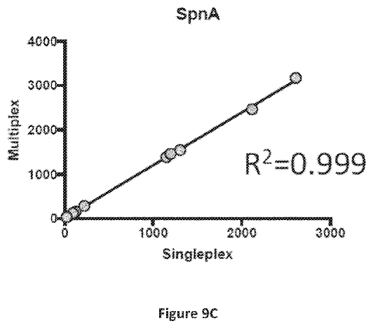

To assess whether the titres to the three antigens could be measured simultaneously the results of singleplex assays were compared with multiplex assays. Singleplex assays were performed in which each bead was incubated with sera from participants, and the same sera were then tested in multiplex assays in which the three antigen beads were mixed in equal parts and incubated with the test sera in a single assay well. The MFI in these multiplex assays showed an extremely strong correlation with the singleplex MFI for each antigen, as shown in FIG. 9, with R² values as follows: SLO=0.999 (FIG. 9A); DNaseB B=0.999 (FIG. 9B); and SpnA=0.999 (FIG. 9C). This indicates there was no interference or IgG cross-reactivity between the beads and demonstrates the feasibility of a multiplex Luminex-based assay comprising the three streptococcal antigens.

The Coefficient of Variability (CV) for the multiplex Luminex assay was assessed using the same 10 sera utilised in the singleplex/multiplex comparison above. The concentrations of IgG for these 10 sera were measured in assays incorporating the IgG standard curves and the average intra- and inter-assay CVs were <2% and <11%, respectively, for each of the antigens (Table 3).

TABLE 3
Coefficients of Variability

| n = 10 | SLO | DNaseB | SpnA |
|---|---|---|---|
| Intra-assay CV (%) | 1.8 | 1.5 | 1.8 |
| Inter-assay CV (%) | 9.3 | 10.4 | 10.4 |

These CVs demonstrate the multiplex Luminex-based assay has good precision and is repeatable. The reproducibility of both the standard curve and the assay test results means these reagents can be utilised to also check the coupling efficacy and integrity of future batches of antigen-coupled beads.

Comparison with Existing Serological Tests

To compare the Luminex assay with existing, commercially available methodology, sera from 61 participants for which sufficient volumes were available were subjected to ASO and ADB testing. ASO was measured as described in Example One above, using the widely employed turbidimetric technique and exact values in international units (IU/mL) were obtained. ADB titres were measured (again as described in Example One above) using an enzyme inhibition assay that provides titre ranges (100, 200, 300, 400, 600, 800, 1200 and 1600). As shown in FIG. 10A, there was a good correlation between the concentration of ASO IgG determined in the commercially available turbidimetric technique and the anti-SLO antibody titre determined by Luminex assay (R²=0.933). As shown in FIG. 10B, there was also a good correlation between the concentration of ADB IgG determined in the commercially available enzyme inhibition assay and the anti-DNaseB antibody titre determined in the Luminex assay (R²=0.942).

### Immunokinetics in ARF

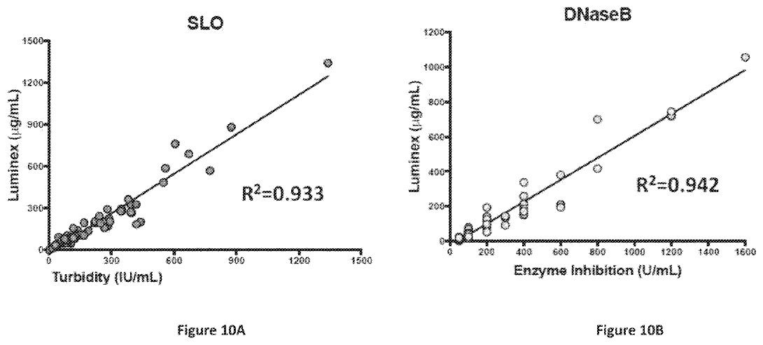

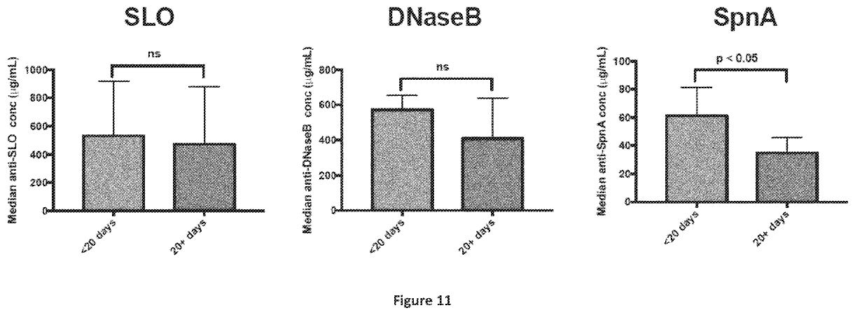

Sera from patients diagnosed with ARF (RFRF study) was stratified by days from hospitalization. As can be readily seen in FIG. 11, levels of anti-SpnA IgG was significantly reduced in sera collected more than 20 days from hospitalization (n=17) compared to levels in sera collected less than 20 days (n=19), indicative of a shorter half-life than either anti-SLO antibodies or anti-DNaseB antibodies.

This in turn suggests that SpnA has favourable immunokinetics for Streptococcal serology, supporting its use in diagnostic analyses, particularly in multiplex assays such as those described and exemplified herein. It will be appreciated that the analyses enabled by the invention disclosed herein allows the rapid identification of and treatment of patients who have been recently exposed to Streptococcus pyogenes, but for whom long-term antibiotic treatment—frequently extending for many years with attendant expense and risk—is unnecessary and can be avoided.

### Example Three

This example describes the further assessment of a bead-based multiplex assay using the Luminex platform to determine the presence, amount and immunokinetics of GAS-specific antibodies in biological samples.

### Methods

### Study Subjects

In this study, human serum was obtained as described in Example One above. Again, all participants had provided written informed consent and appropriate ethical board approval was obtained.

### Serum Preparation

Serum samples from patients with acute rheumatic fever were grouped by the day blood samples were obtained: less than 20 days from hospital admission date; 20 days or greater after hospital admission. Sera samples were diluted 1:15,000 for analysis. The IVIG reference was diluted 1:60,000 prior to use.

### Luminex Bead Preparation and Assay

Each of the antigen:xMAP microspheres for use in the assay were prepared as described in Example Two. Luminex assays were conducted on a Magpix™ system (Merck), in accordance with the manufacturer's instructions.

Comparison of serum antibody titres for SLO, DnaseB and SpnA was determined using the triplex Luminex assay. Statistical analysis was performed using GraphPad Prism 7.0 software.

### Results

### Immunokinetics in ARF

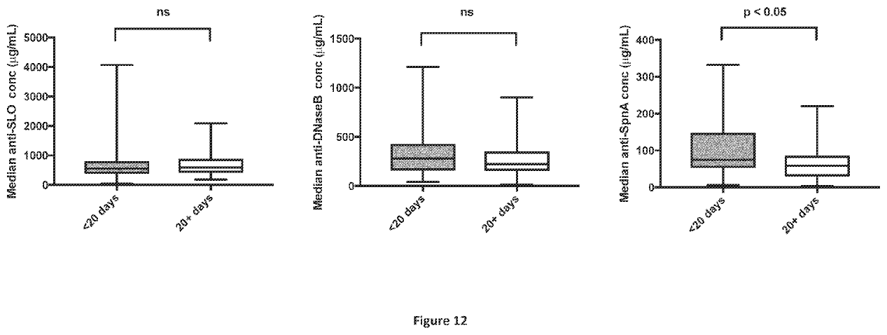

Sera from patients diagnosed with ARF (RFRF study) was stratified by days from hospitalization. The results of this analysis are shown in Table 4 below and FIG. 12.

TABLE 4
Immunokinetics

| | <20 days | 20+ days | |
|---|---|---|---|
| Antigen | (n = 48) | (n = 37) | p-value |
| SLO | | | |
| mean (95% CI) | 834.9 (598.3-1072) | 695.2 (552.1-838.4) | |
| median (95% CI) | 558.1 (430.9-740.6) | 592.0 (465-727.8) | ns (p = 0.8983) |
| Dnase B | | | |
| mean (95% CI) | 341.2 (267.7-414.6) | 288.1 (220-356.2) | |
| median (95% CI) | 279.6 (238.2-355.7) | 223.3 (184.2-293) | ns (p = 0.3248) |
| SpnA | | | |
| mean (95% CI) | 103.4 (79.4 -127.5) | 70.1 (51.7-88.5) | |
| median (95% CI) | 75.1 (61.2-91.6) | 59.3 (43.0-69.8) | p = 0.0389 |

There was no significant difference in antibody titres for SLO and DnaseB between these groups. However, as can be readily seen in FIG. 12, levels of anti-SpnA IgG were significantly reduced in sera collected more than 20 days from hospitalization (n=37) compared to levels in sera collected less than 20 days from hospital admission (n=48), consistent with the results presented in Example Two above. This supports the conclusion that anti-SpnA antibodies have a shorter half-life than either anti-SLO antibodies or anti-DNaseB antibodies.

This in turn suggests that SpnA has favourable immunokinetics for Streptococcal serology, supporting its use in diagnostic analyses (particularly in multiplex assays such as those described and exemplified herein), therapeutic assessments, and treatment regimens.

### Example Four

This example describes the characterisation of SpnA thermostability as part of an assessment for suitability for use in diagnostic assays, for example in a bead-based multiplex assay such as the Luminex platform, or on dipstrip/dipstick assays (particularly in circumstances where cold chain storage is not practical or available).

### Methods

The stability at room temperature of the full-length SpnA (aa28-877) and of the C-terminally truncated SpnA (aa28-854, SEQ ID No. 8) was determined. Briefly, aliquots of each protein were stored at room temperature for 5 days. Proteins were visualised using SDS-PAGE and compared with protein that was not stored at room temperature for any length of time (0 days).

### Results

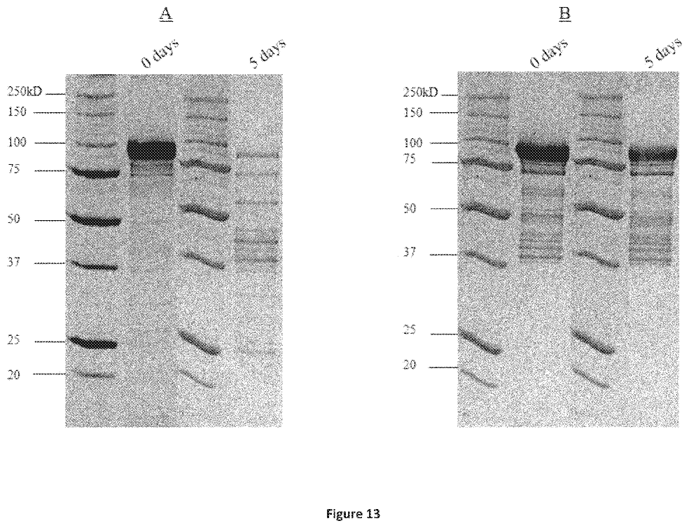

Intact SpnA is present for both full length and C-terminally truncated polypeptides as a band at ˜85 kDa. The full-length construct, however, is less stable at room temperature as shown by the marked reduction in the amount of the ˜85 kDa band after 5 days incubation (FIG. 13A). In contrast, there is minimal change in the amount of ˜85 kDa protein for the truncated SpnA after 5 days incubation at room temperature, as shown in FIG. 13B.

These data clearly show the greater stability of the truncated SpnA polypeptide when stored at room temperature, compared to the full length, native SpnA polypeptide. These results support the use of the truncated SpnA in diagnostic assays, therapeutic assessments, and treatment regimens, particularly in circumstances where long term storage of reagents, or storage under conditions that would be non-optimal for typical protein-based compositions (e.g., at room temperature or in the absence of cold chain).

### Example Five

This example describes the characterisation of SpnA thermostability as part of an assessment for suitability for use in diagnostic assays, for example in a bead-based multiplex assay such as the Luminex platform, or on dipstrip/dipstick assays (particularly in circumstances where cold chain storage is not practical or available).

### Methods

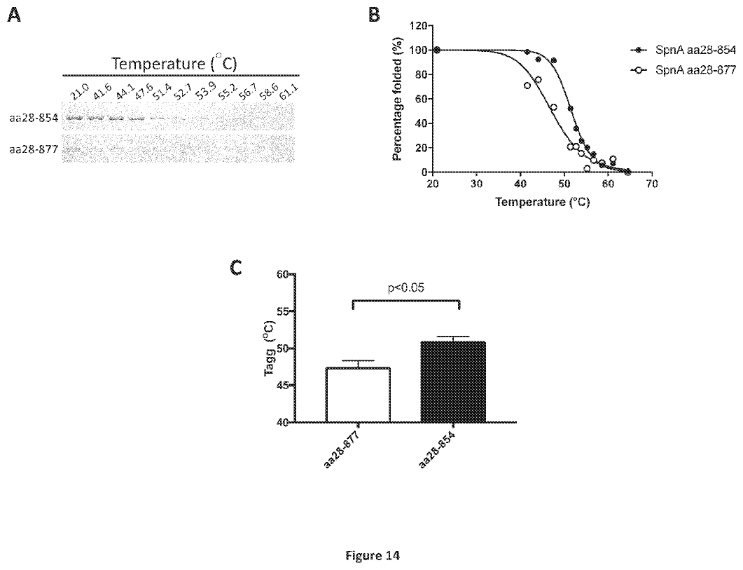

SpnA stability was determined using thermal melts as per published protocols (Moreau, M. J. J., Morin, I. & Schaeffer, P. M. Mol. BioSyst. 6, 1285-1292 (2010)). Briefly, the thermal stability of the original SpnA construct (aa28-877) was compared to the truncated construct (aa28-854, SEQ ID No. 8) by incubating the proteins in identical buffers for 5 minutes at the temperatures shown in FIG. 14A.

This was followed by a cooling and centrifugation step to remove protein aggregates. The percentage folded protein was determined by SDS-PAGE, and the Tagg values (temperature at which 50% of proteins are aggregated) from the thermal aggregation profiles were calculated using GraphPad Prism 7.0 software.

### Results

The percentage of folded protein at each temperature as assessed by SDS-PAGE is shown in the chromatograph of FIG. 14A. The Tagg (temperature at which 50% of proteins are aggregated) for each protein at each temperature was plotted (FIG. 14B), which clearly shows the differences in the melt curves of these two proteins. The mean Tagg value for the truncated construct, 51.0+/−0.6° C. across three replicate experiments, was significantly higher than for the original construct, being 47.5+/−0.9° C., with p<0.05, as can clearly be seen in FIG. 14C.

This clearly shows the greater thermostability of the truncated SpnA polypeptide, supporting its use in diagnostic assays, therapeutic assessments, and treatment regimens, particularly in circumstances where long term storage of reagents, or storage under conditions that would be non-optimal for typical protein-based compositions, such as the absence of cold chain.

### PUBLICATIONS

1. Johnson D R, Kurlan R, Leckman J, Kaplan E L. The human immune response to streptococcal extracellular antigens: clinical, diagnostic, and potential pathogenetic implications. Clin. Infect. Dis. 2010; 50:481-90.
2. Chang A, Khemlani A, Kang H, Proft T. Functional analysis of Streptococcus pyogenes nuclease A (SpnA), a novel group A streptococcal virulence factor. Molecular Microbiology. 2011; 79:1629-42.
3. Atatoa-Carr P, Lennon D, Wilson N, New Zealand Rheumatic Fever Guidelines Writing Group. Rheumatic fever diagnosis, management, and secondary prevention: a New Zealand guideline. N Z Med J. 2008; 121:59-69.
4. Raynes J M, Frost H R C, Williamson D A, Young P G, Baker E N, steemson J D, et al. Serological Evidence of Immune Priming by Group A Streptococci in Patients with Acute Rheumatic Fever. Front Microbiol. 2016; 7:1119.
5. Dabitao D, Margolick J B, Lopez J, Bream J H. Multiplex measurement of proinflammatory cytokines in human serum: comparison of the Meso Scale Discovery electrochemiluminescence assay and the Cytometric Bead Array. J Immunol Methods. 2011; 372:71-7.
6. Steer A C, Vidmar S, Ritika R, Kado J, Batzloff M, Jenney A W J, et al. Normal ranges of streptococcal antibody titers are similar whether streptococci are endemic to the setting or not. Clin. Vaccine Immunol. 2009; 16:172-5.
7. Moreau, M. J. J., Morin, I. & Schaeffer, P. M. Mol. BioSyst. 6, 1285-1292 (2010).

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the present invention have been described by way of example only and it should be appreciated that modifications and additions may be made thereto without departing from the scope thereof.

1.-54. (canceled)

55. A method of treating a patient suffering from rheumatic fever or PSGN, the method comprising the steps of:
i) providing a biological sample from the subject that is capable of or suspected of containing one or more antibodies specific for Streptococcus pyogenes; and
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) assessing one or more other diagnostic criteria for rheumatic fever or PSGN in the subject;
iv) wherein the presence of Streptococcus pyogenes SpnA antigen-specific complexes, or detection of an amount of Streptococcus pyogenes SpnA antigen-specific complexes above a threshold value, is indicative of a recent exposure to Streptococcus pyogenes in the subject;
v) and wherein the presence of one or more other diagnostic criteria for rheumatic fever or PSGN, together with the absence of Streptococcus pyogenes SpnA antigen-specific complexes, or the detection of an amount of Streptococcus pyogenes SpnA antigen-specific complexes below a threshold value, is indicative of a prior exposure to Streptococcus pyogenes in the subject; and
vi) if the subject has had a recent exposure to Streptococcus pyogenes, then administering treatment for recent-onset rheumatic fever or acute PSGN; and if the subject has had a prior exposure to Streptococcus pyogenes, then administering treatment for established or subsequent Streptococcus pyogenes infection, or administering treatment for rheumatic fever or PSGN.

56. The method of claim 55, wherein assessing one or more other diagnostic criteria comprises the steps of:
i) contacting the biological sample with one or more populations of an antigen from Streptococcus pyogenes DNaseB and/or one or more populations of an antigen from Streptococcus pyogenes SLO, wherein the one or more populations of Streptococcus pyogenes antigen is capable of binding antigen-specific antibodies present in the biological sample to form one or more populations of antigen:antigen-specific antibody complexes if the antigen-specific antibodies are present in the biological sample; and
ii) detecting the complexes, wherein
a. the presence of Streptococcus pyogenes SpnA-specific complexes, or the detection of an amount of Streptococcus pyogenes SpnA-specific complexes above a threshold value, is indicative of a recent exposure to Streptococcus pyogenes in the subject, and
b. the presence of Streptococcus pyogenes DNaseB-specific complexes and/or of Streptococcus pyogenes SLO-specific complexes, and the absence of Streptococcus pyogenes SpnA-specific complexes or the detection of an amount of Streptococcus pyogenes SpnA-specific complexes below a threshold value, is indicative of a prior exposure to Streptococcus pyogenes in the subject,
and wherein
if the subject has had a recent exposure to Streptococcus pyogenes, the treatment for recent-onset rheumatic fever or acute PSGN comprises administering an antibiotic effective against acute Streptococcus pyogenes infection; and if the subject has had a prior exposure to Streptococcus pyogenes, the treatment for established or subsequent Streptococcus pyogenes infection or for rheumatic fever or PSGN comprises administering an antibiotic effective against established or subsequent Streptococcus pyogenes infection.

57. The method of claim 55 wherein the treatment for recent-onset rheumatic fever or acute PSGN, or treatment for recent Streptococcus pyogenes infection, is administration of an antibiotic effective against acute Streptococcus pyogenes infection according to a treatment regimen comprising administration of a loading dose of said antibiotic over an acute treatment period.

58. The method of claim 56, wherein the antibiotic is bicillin.

59. A method for detecting recent exposure to or the presence of Streptococcus pyogenes in a subject, the method comprising:
i) providing a biological sample from the subject that is capable of or suspected of containing antibodies specific for one or more Streptococcus pyogenes antigens;
ii) contacting the biological sample with one or more populations of an antigen from Streptococcus pyogenes SpnA, wherein the one or more populations of Streptococcus pyogenes SpnA antigen is capable of binding antigen-specific antibodies present in the biological sample to form one or more populations of antigen:antigen-specific antibody complexes if the antigen-specific antibodies are present in the biological sample; and
iii) detecting the complexes, wherein an increase in detection of one or more complexes above a threshold value is indicative of a recent exposure to or the presence of Streptococcus pyogenes in the subject.

60. The method of claim 59, additionally comprising assessing one or more diagnostic criteria for rheumatic fever or poststreptococcal glomerulonephritis (PSGN) including acute poststreptococcal glomerulonephritis (APSGN) in a subject, or an increased likelihood of developing rheumatic fever or PSGN in a subject,
i) wherein an increase in detection of one or more complexes above a threshold value is indicative of an increased likelihood of developing rheumatic fever or APSGN, or is indicative of a recent exposure to Streptococcus pyogenes in the subject as one criteria for the presence of rheumatic fever or PSGN in the subject;
ii) and wherein an increase in detection of one or more complexes above a threshold value in conjunction with one or more other diagnostic criteria for rheumatic fever or APSGN is indicative of rheumatic fever or PSGN in the subject.

61. The method of claim 59 wherein the increase in detection of one or more complexes is an increase relative to a reference level of the antigen established for each test population.

62. The method of claim 60 wherein the one or more diagnostic criteria is the presence or absence of one or more clinical symptoms associated with rheumatic fever or PSGN.

63. The method of claim 62 wherein the one or more clinical symptoms are selected from migratory polyarthritis, carditis, hematuria, erythema marginatum, subcutaneous nodules, Seydenham's Chorea, or pyoderma.

64. The method of claim 55 wherein one or more of the Streptococcus pyogenes antigens is an antigen from one of the following proteins:
i) Streptococcus pyogenes nuclease A (SpnA),
ii) Deoxyribonuclease-B (DNaseB), or
iii) Streptolysin-O (SLO).

65. The method of claim 60, wherein assessing one or more other diagnostic criteria comprises the steps of:
i) contacting the biological sample with one or more populations of an antigen from Streptococcus pyogenes DNaseB and/or one or more populations of an antigen from Streptococcus pyogenes SLO, wherein the one or more populations of Streptococcus pyogenes antigen is capable of binding antigen-specific antibodies present in the biological sample to form one or more populations of antigen:antigen-specific antibody complexes if the antigen-specific antibodies are present in the biological sample; and
ii) detecting the complexes, wherein
a. the presence of Streptococcus pyogenes SpnA-specific complexes, or the detection of an amount of Streptococcus pyogenes SpnA-specific complexes above a threshold value, is indicative of a recent exposure to Streptococcus pyogenes in the subject, and
b. the presence of Streptococcus pyogenes DNaseB-specific complexes and/or of Streptococcus pyogenes SLO-specific complexes, and the absence of Streptococcus pyogenes SpnA-specific complexes or the detection of an amount of Streptococcus pyogenes SpnA-specific complexes below a threshold value, is indicative of a prior exposure to Streptococcus pyogenes in the subject.

66. The method of claim 59 wherein one or more of the Streptococcus pyogenes antigens is selected from the group consisting of:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8, or
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5, or
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2, or
x) any combination of two or more of i) to ix) above.

67. The method of claim 66 wherein the biological sample is contacted with a population of each of the following Streptococcus pyogenes antigens:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8,
and
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5,
and
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2.

68. The method of claim 66 wherein the biological sample is contacted with a population of each of the following Streptococcus pyogenes antigens:
i) Streptococcus pyogenes nuclease A (SpnA),
ii) Deoxyribonuclease-B (DNaseB), and
iii) Streptolysin-O (SLO).

69. The method of claim 66 wherein the biological sample is contacted with a population of each of the following Streptococcus pyogenes antigens:
i) an antigenic fragment of SpnA comprising, consisting essentially of, or consisting of the amino acid sequence of SEQ ID No. 8,
ii) an antigenic fragment of DNaseB comprising, consisting essentially of, or consisting of the amino acid sequence of SEQ ID No. 5, and
iii) an antigenic fragment of SLO comprising, consisting essentially of, or consisting of the amino acid sequence of SEQ ID No. SEQ ID No. 1 or SEQ ID No. 2.

70. The method of claim 59, wherein detecting the antigen:antibody complexes comprises exposing the complexes to a specific binding partner that carries a detectable label and detecting a signal from the label if the antigen-specific antibodies are present in the biological sample.

71. The method of claim 70 wherein the specific binding partner comprises an antibody or fragment thereof.

72. The method of claim 59, wherein the antigen:antibody complexes are detected is by an ELISA or a luminex assay or using a flow instrument, a plate-based immunological assay, electrophoresis and/or immunoblot, an immunochromatographic strip, an electronic biosensor, a resonance biosensor, or a microfluidic device or sensor.

73. The method of claim 59 wherein one or more of the Streptococcus pyogenes antigens are detectably labelled.

74. A kit for detecting or diagnosing rheumatic fever or PSGN in a subject, for detecting the presence of Streptococcus pyogenes infection in a subject, or for detecting Streptococcus pyogenes antigen-specific antibodies in a biological sample, the kit comprising a composition comprising at least one of the Streptococcus pyogenes antigens selected from the group consisting of:
i) Streptococcus pyogenes nuclease A (SpnA), or
ii) an antigenic fragment of SpnA comprising, consisting essentially of, or consisting of the amino acid sequence of SEQ ID No. 8, or
iii) an antigenic fragment of SpnA comprising, consisting essentially of, or consisting of at least 10 contiguous amino acids from the amino acid sequence of SEQ ID No. 8,
and optionally at least one of the Streptococcus pyogenes antigens selected from the group consisting of:
iv) Deoxyribonuclease-B (DNaseB), or
v) an antigenic fragment of DNaseB comprising, consisting essentially of, or consisting of the amino acid sequence of SEQ ID No. 5, or
vi) an antigenic fragment of DNaseB comprising, consisting essentially of, or consisting of at least 10 contiguous amino acids from the amino acid sequence of SEQ ID No. 5, or
vii) Streptolysin-O (SLO), or
viii) an antigenic fragment of SLO comprising, consisting essentially of, or consisting of the amino acid sequence of SEQ ID No. 1 or SEQ ID No. 2, or
ix) an antigenic fragment of SLO comprising, consisting essentially of, or consisting of at least 10 contiguous amino acids from the amino acid sequence of SEQ ID No. 1 or SEQ ID No. 2,
optionally at least one composition comprising a reference antibody control, wherein the antibody control comprises an antibody specific to one of the Streptococcus pyogenes antigens present in the kit,
optionally one or more reagents for constituting the medium favourable for contacting the one or more antigens with a biological sample,
optionally one or more reagents enabling the detection of a complex formed between the one or more antigens and one or more Streptococcus pyogenes antigen specific antibodies present in a biological sample,
and instructions for use.
